(19) United States
(12) Patent Application Publication
(10) Pub. No.: US 2024/0188842 A1
(43) Pub. Date: Jun. 13, 2024

(54) FLUID STATUS MONITORING

(71) Applicant: WearOptimo Pty Ltd, West End, Queensland (AU)

(72) Inventors: Anthony Mark BREWER, West End (AU); Farrukh MATEEN, West End (AU); Stephen James WILSON, West End (AU)

(21) Appl. No.: 18/286,739

(22) PCT Filed: Apr. 11, 2022
(86) PCT No.: PCT/AU2022/050322

§ 371 Date: Oct. 12, 2023

(30) Foreign Application Priority Data
Apr. 13, 2021 (AU) 2021901075

(51) Int. Cl. A61B 5/0537; A61B 5/00; A61B 5/0538; A61B 5/145

(52) U.S. Cl. CPC A61B 5/0537; A61B 5/0538; A61B 5/14514; A61B 5/4266; A61B 5/6832; A61B 5/685; A61B 2562/0209; A61B 2562/028; A61B 2562/046

(57) ABSTRACT

A system for monitoring a fluid status of a biological subject, the system including at least one substrate including a plurality of microstructures including electrodes configured to breach a stratum corneum of the subject, a signal generator configured to apply an electrical stimulatory signal between electrodes on different microstructures, at least one signal sensor configured to measure electrical response signals between electrodes on different microstructures and one or more electronic processing devices that are configured to determine changes in bioimpedance using the measured electrical response signals and analyse the changes in bioimpedance to determine at least one indicator at least partially indicative of the fluid status of the subject.

### BACKGROUND OF THE INVENTION

The present invention relates to a system and method for performing measurements on a biological subject, and in one particular example, to performing measurements of fluid levels on a biological subject by breaching a stratum corneum of the subject using microstructures to thereby perform fluid status monitoring.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Water is essential for all forms of life. Without it, a person can only survive days. Comprising 75% of the body by weight (dependent on age), water plays a variety of roles in the body homeostasis. Thermoregulation through sweat and conductive heat loss via vasodilation rely on the evaporative cooling properties and specific heat of water, respectively.

Regulation of water is a key homeostatic requirement in the human. Oral ingestion, insensible losses (urine, faeces) and sweat loss are balanced through the tightly regulated control of plasma osmolarity and blood volume. The sensation of thirst drives oral water intake when it is available, but body water losses may outstrip water intake in heat-stressed environments, particularly in active military activities where extreme physical exertion may be required and water availability is absent or limited.

Failure to maintain adequate body water, through imbalance in water intake versus water losses will lead to dehydration and a concomitant plasma osmolarity increase. The deleterious effects of dehydration are seen across physical performance, cognitive function, and permanent end-organ damage or death. In order to address these risks, normal human physiology provides a feedback control system whereby increases in plasma osmolarity trigger the centrally mediated thirst sensation, however thirst is relatively insensitive in acutely tracking fluid status under exertion. The maintenance of hydration during physical exertion is further compromised due to the availability of fluids and the relative under-perfusion of the gut, which will reduce the rate of water uptake into plasma. Involuntary dehydration to the point of 2-3% of body mass during physical exertion is therefore commonplace and may trigger precautionary voluntary over-hydration behaviour in some individuals leading to health risks due to electrolyte dilution (hyponatraemia) and can result in death. Body water assessment remains a clinical measurement issue with no clear consensus as to the best laboratory test or index. In the field, body water loss assessments are further compromised and body weighing, urine specific gravity skin turgor and sweat detection provide inadequate solutions.

Surface-based sweat detection and analysis and whole body bioimpedance approaches have been relatively recent candidate technologies for monitoring hydration. Sweat based measures are compromised by the idiosyncratic nature of sweat content and the nonuniform distribution of eccrine sweat glands. Impedance measurements typically utilise surface-based electrodes to apply a current through tissue, with an electrical potential across the tissue being measured and used to derive an impedance measurement. Analysis of the impedance measurement can then be used to derive information regarding fluid levels in the subject, such as levels of intra-cellular and/or extra-cellular. Whole body bioimpedance analysis relies on multi-frequency electrical interrogation of the body's tissues (muscles, skin, bone, blood, air). Differing electrical properties of tissue types such as fat, muscle, bone, air and blood are interrogated by impedance measures over a range of frequencies. While non-invasive, it is heavily reliant on population derived parameters such as age, gender, body size and limb length, and also is adversely affected by sweat.

US20110295100 describes methods, systems and/or devices for enhancing conductivity of an electrical signal through a subject's skin using one or more microneedle electrodes are provided. A microneedle electrode may be applied to the subject's skin by placing the microneedle electrode in direct contact with the subject's skin. The microneedles of the microneedle electrode may be inserted into the skin such that the microneedles pierce stratum corneum of the skin up to or through dermis of the skin. An electrical signal passes or is conducted through or across the microneedle electrode and the subject's skin, where impedance of the microneedle electrode is minimal and greatly reduced compared to existing technologies.

US 2019/0013425 describes a biometric information measuring sensor is provided that includes a base comprising a plurality of bio-marker measuring areas and a plurality of electrodes. Each of the plurality of electrodes is disposed on a respective one of the plurality of bio-marker measuring areas, and each of the plurality of electrodes includes a working electrode and a counter electrode spaced apart from the working electrode. The biometric information measuring sensor also includes a plurality of needles. Each of the needles is disposed on a respective one of the plurality of electrodes. Two or more of the plurality of needles have different lengths.

US20150208984 describes a transdermal microneedle continuous monitoring system. The continuous system monitoring includes a substrate, a microneedle unit, a signal processing unit and a power supply unit. The microneedle unit at least comprises a first microneedle set used as a working electrode and a second microneedle set used as a reference electrode, the first and second microneedle sets arranging on the substrate. Each microneedle set comprises at least a microneedle. The first microneedle set comprises at least a sheet having a through hole on which a barbule forms at the edge. One of the sheets provides the through hole from which the barbules at the edge of the other sheets go through, and the barbules are disposed separately.

U.S. Pat. No. 8,588,884 describes devices for enhancing conductivity of an electrical signal through a subject's skin using one or more microneedle electrodes are provided. A microneedle electrode may be applied to the subject's skin by placing the microneedle electrode in direct contact with the subject's skin. The microneedles of the microneedle electrode may be inserted into the skin such that the microneedles pierce stratum corneum of the skin up to or through dermis of the skin. An electrical signal passes or is conducted through or across the microneedle electrode and the subject's skin, where impedance of the microneedle electrode is minimal and greatly reduced compared to existing technologies.

WO2020069565 describes a system for performing measurements on a biological subject, the system including: at least one substrate including a plurality of plate microstructures configured to breach a stratum corneum of the subject; at least one sensor operatively connected to at least one microstructure, the at least one sensor being configured to measure response signals from the at least one microstructure; and, one or more electronic processing devices configured to: determine measured response signals; and, at least one of: provide an output based on measured response signals; perform an analysis at least in part using the measured response signals; and, store data at least partially indicative of the measured response signals.

### SUMMARY OF THE PRESENT INVENTION

In one broad form, an aspect of the present invention seeks to provide a system for monitoring a fluid status of a biological subject, the system including: at least one substrate including a plurality of microstructures including electrodes configured to breach a stratum corneum of the subject; a signal generator configured to apply an electrical stimulatory signal between electrodes on different microstructures; at least one signal sensor configured to measure electrical response signals between electrodes on different microstructures; and, one or more electronic processing devices that are configured to: determine changes in bioimpedance using the measured electrical response signals; and, analyse the changes in bioimpedance to determine at least one indicator at least partially indicative of the fluid status of the subject.

In one embodiment the bioimpedance is at least one of: measured at a single frequency; measured at multiple different frequencies; and, derived from impedance measurements performed at multiple different frequencies.

In one embodiment the bioimpedance is indicative of at least one of: intracellular fluid levels; extracellular fluid levels; and, blood fluid levels.

In one embodiment the change in bioimpedance includes at least one of: a change in a bioimpedance magnitude; a change in a bioimpedance phase angle; a change in intracellular fluid levels; a change in extracellular fluid levels; and, a change in blood fluid levels.

In one embodiment the one or more electronic processing devices are configured to: analyse changes in bioimpedance to determine fluid movement between fluid compartments; and, generate the indicator based on the determined fluid movement.

In one embodiment the one or more electronic processing devices are configured to: determine a baseline bioimpedance; and, analyse changes in bioimpedance relative to the baseline bioimpedance.

In one embodiment the one or more electronic processing devices are configured to: determine a perturbation event that will perturb fluid levels in the subject; and, analyse the changes in bioimpedance at least in part in accordance with the perturbation event.

In one embodiment the perturbation event includes at least one of: a change in physical activity state; a change in posture; heating; cooling; ingestion of fluid; administration of medication; administration of a pharmacological agent; a medical procedure; dialysis; administration of intravenous fluids; administration of intravenous blood; onset of illness or disease; and, a physiological perturbation.

In one embodiment the one or more electronic processing devices are configured to at least one of: determine a change in bioimpedance measured before and after the perturbation event; determine a change in bioimpedance measured during the perturbation event; determine a change in bioimpedance during a time period after the perturbation event; and, determine a rate of change in bioimpedance during a time period after the perturbation event.

In one embodiment the one or more electronic processing devices are configured to: compare multiple changes in bioimpedance, each change in bioimpedance being associated with a respective perturbation event; and, determine the indicator based on the multiple changes in bioimpedance.

In one embodiment the one or more electronic processing devices are configured to: determine a gradient of a rate of change in bioimpedance after each of multiple perturbation events; and, determine the indicator based on the changes in the gradients.

In one embodiment the one or more electronic processing devices are configured to determine the perturbation event based on at least one of: user input commands; signals from at least one sensor; changes in a subject movement; changes in a subject posture; changes in a subject temperature; changes in a subject heart rate; changes in a subject respiratory rate; and, changes in a subject blood oxygen levels.

In one embodiment the system includes a sensor at least one of: mounted on the substrate; and, provided within a housing attached to the substrate, and wherein the one or more processing devices are configured to: monitor sensor signals from the at least one sensor; and, determine the perturbation event in accordance with the sensor signals.

In one embodiment the indicator is indicative of at least one of: over hydration; under hydration; normal hydration; restoration; trending towards dehydration; and, maldistribution of fluid between compartments.

In one embodiment at least one of: the microstructures are arranged in pairs and wherein the bioimpedance is measured using at least one of: multiple pairs of electrodes; and, pairs of electrodes with different spacings; and, the microstructures are arranged in rows and wherein the bioimpedance is measured between at least one of: electrodes on different rows of microstructures; and, electrodes on different rows of microstructures with different spacings.

In one embodiment at least some of the microstructures are blade microstructures.

In one embodiment a spacing between the microstructures is at least one of: about 2 mm; about 1 mm; about 0.5 mm; about 0.2 mm; and, about 0.1 mm.

In one embodiment at least some of the microstructures at least one of: are at least partially tapered and have a substantially rounded rectangular cross sectional shape; have a length that is at least one of: less than 300 μm; about 150 μm; greater than 100 μm; and, greater than 50 μm; have a maximum width that is at least one of: of a similar order of magnitude to the length; greater than the length; about the same as the length; less than 300 μm; about 150 μm; and, greater than 50 μm; and, have a thickness that is at least one of: less than the width; significantly less than the width; of a smaller order of magnitude to the length; less than 100 μm; about 25 μm; and, greater than 10 μm.

In one embodiment at least some of the microstructures have a tip that at least one of: has a length that is at least one of: less than 50% of a length of the microstructure; at least 10% of a length of the microstructure; and, about 30% of a length of the microstructure; and, has a sharpness of at least one of: at least 0.1 μm; less than 5 μm; and, about 1 μm.

In one embodiment at least some of the microstructures include at least one of: a shoulder that is configured to abut against the stratum corneum to control a depth of penetration; a shaft extending from a shoulder to the tip, the shaft being configured to control a position of the tip in the subject; and, anchor microstructures used to anchor the substrate to the subject.

In one embodiment the microstructures have a density that is at least one of: less than 5000 per cm²; greater than 100 per cm²; and, about 600 per cm².

In one embodiment the substrate includes electrical connections to allow electrical signals to be applied to and/or received from respective microstructures.

In one embodiment the system includes one or more switches for selectively connecting at least one of the at least one sensor and at least one signal generator to one or more of the microstructures and wherein the one or more processing devices are configured to control the switches and the signal generator to allow at least one measurement to be performed.

In one embodiment the system includes: a substrate coil positioned on the substrate and operatively coupled to one or more microstructure electrodes; and, an excitation and receiving coil positioned in proximity to the substrate coil such that alteration of a drive signal applied to the excitation and receiving coil acts as a response signal.

In one embodiment the microstructures include an insulating layer extending over at least one of: part of a surface of the microstructure; a proximal end of the microstructure; at least half of a length of the microstructure; about 90 μm of a proximal end of the microstructure; and, at least part of a tip portion of the microstructure.

In one embodiment at least one electrode at least one of: has a surface area of at least one of: less than 200,000 μm²; about 22,500 μm²; and, at least 2,000 μm²; extends over a length of a distal portion of the microstructure; extends over a length of a portion of the microstructure spaced from the tip; is positioned proximate a distal end of the microstructure; is positioned proximate a tip of the microstructure; extends over at least 25% of a length of the microstructure; extends over less than 50% of a length of the microstructure; extends over about 60 μm of the microstructure; and, is configured to be positioned in a viable epidermis of the subject in use.

In one embodiment the microstructures include a material including at least one of: a material to reduce biofouling; a material to attract at least one substance to the microstructures; and, a material to repel at least one substance from the microstructures.

In one embodiment at least some of the microstructures are coated with a coating and wherein the coating at least one of: modifies surface properties to at least one of: increase hydrophilicity; increase hydrophobicity; and, minimize biofouling; attracts at least one substance to the microstructures; repels at least one substance from the microstructures; acts as a barrier to preclude at least one substance from the microstructures; and, includes at least one of: a permeable membrane; polyethylene; polyethylene glycol; polyethylene oxide; zwitterions; peptides; hydrogels; and, self-assembled monolayer.

In one embodiment the system includes: a patch including the substrate and microstructures; and, a monitoring device that is configured to: perform the measurements; and, at least one of: provide an output indicative of the indicator; and, provide a recommendation based on the indicator.

In one embodiment the monitoring device is at least one of: inductively coupled to the patch; attached to the patch; and, brought into contact with the patch when a reading is to be performed.

In one embodiment the system includes: a transmitter that transmits at least one of: subject data derived from the measured response signals; and, measured response signals; and, a processing system that: receives subject data derived from the measured response signals; and, analyses the subject data to generate at least one indicator, the at least one indicator being at least partially indicative of a health status associated with the subject.

In one embodiment the system is configured to perform impedance measurements in the viable epidermis to determine an indicator indicative of at least one of: a hydration of the subject; interstitial fluid levels; a change in interstitial fluid levels; an ion concentration in interstitial fluid; a change in an ion concentration in interstitial fluid; an ion concentration; a change in an ion concentration; a total body water; intracellular fluid levels; extracellular fluid levels; plasma water levels; fluid volumes; and, hydration levels.

In one broad form, an aspect of the present invention seeks to provide a method for monitoring a fluid status of a biological subject, the method including: providing: at least one substrate including a plurality of microstructures including electrodes configured to breach a stratum corneum of the subject; a signal generator configured to apply an electrical stimulatory signal between electrodes on different microstructures; and, at least one signal sensor configured to measure electrical response signals between electrodes on different microstructures; and, using one or more electronic processing devices to: determine changes in bioimpedance using the measured electrical response signals; and, analyse the changes in bioimpedance to determine at least one indicator at least partially indicative of the fluid status of the subject.

In one broad form, an aspect of the present invention seeks to provide a system for monitoring a fluid status of a biological subject, the system including: at least one substrate including a plurality of microstructures including electrodes configured to breach a stratum corneum of the subject; a signal generator configured to apply an electrical stimulatory signal between electrodes on different microstructures; at least one signal sensor configured to measure electrical response signals between electrodes on different microstructures; and, one or more electronic processing devices that are configured to: determine one or more bioimpedance values using the measured electrical response signals; and, analyse the one or more bioimpedance values to determine at least one indicator at least partially indicative of the fluid status of the subject.

In one broad form, an aspect of the present invention seeks to provide a method for monitoring a fluid status of a biological subject, the method including: providing: at least one substrate including a plurality of microstructures including electrodes configured to breach a stratum corneum of the subject; a signal generator configured to apply an electrical stimulatory signal between electrodes on different microstructures; and, at least one signal sensor configured to measure electrical response signals between electrodes on different microstructures; and, using one or more electronic processing devices to: determine one or more bioimpedance values using the measured electrical response signals; and, analyse the one or more bioimpedance values to determine at least one indicator at least partially indicative of the fluid status of the subject.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:—

FIG. 1 is a schematic diagram of an example of a system for performing measurements on a biological subject;

FIG. 2 is a flow chart of an example of a process for performing measurements on a biological subject;

FIG. 3A is a schematic side view of a further example of a system for performing measurements on a biological subject;

FIG. 3B is a schematic underside view of an example of a patch for the system of FIG. 3A;

FIG. 3C is a schematic plan view of the patch of FIG. 3B;

FIG. 3D is a schematic side view of the patch of FIG. 3B illustrating depth of penetration of current paths;

FIGS. 3E and 3F are graphs illustrating an example of modelled changes in electrical current density for different spacings of blade microstructures at 1 kHz and 1 MHz, respectively;

FIGS. 3G and 3H are graphs illustrating an example of modelled changes in electrical current density for different spacings of blade microstructures for no sweat and sweat conditions, respectively;

FIGS. 31 and 3J are graphs illustrating an example of modelled changes in electrical current density for different surface electrodes spacings for no sweat and sweat conditions, respectively;

FIG. 4A is a schematic side view of an example of a plate microstructure;

FIG. 4B is a schematic front view of the microstructure of FIG. 4A;

FIG. 4C is a schematic underside view of an example of a patch including the microstructure of FIG. 4A;

FIG. 4D is a schematic perspective topside view of an example of substrate including pairs of blade microstructures of FIGS. 4A and 4B;

FIG. 4E is a schematic plan view of an example of a hexagonal grid microstructure array;

FIG. 4F is a schematic plan view of an alternative example of a grid of pairs of microstructures;

FIG. 4G is a schematic perspective view of an example of a grid of pairs of microstructures;

FIG. 4H is a schematic plan view of the grid of FIG. 4I showing example connections;

FIG. 4I is an image of an example of a patch including arrays of pairs of angularly offset plate microstructures;

FIG. 4J is a schematic side view of a specific example of a plate microstructure;

FIG. 4K is a schematic perspective view of the plate microstructure of FIG. 4J;

FIG. 4L is a schematic side view of an example of a pair of microstructures inserted into a subject for epidermal measurement;

FIG. 4M is a schematic side view of an example of a pair of microstructures inserted into a subject for dermal measurement;

FIG. 4N is an image of an example of a patch including rows of pairs of plate microstructures mounted on mesas;

FIG. 4O is a second image of an example of the patch of FIG. 4N;

FIG. 4P is a schematic perspective view of the patch of FIG. 4N;

FIG. 4Q is a schematic end view of a row of pairs of microstructures of the patch of FIG. 4N;

FIG. 4R is an image of results of a penetration experiment using the patch of FIG. 4N;

FIG. 5 is a flow chart of an example of a process for monitoring hydration;

FIG. 6A is a graph illustrating a change in bioimpedance measured using microstructure electrodes that penetrate the stratum corneum;

FIG. 6B is a graph illustrating a change in bioimpedance measured using skin surface electrodes;

FIGS. 7A to 7S are graphs illustrating a change in bioimpedance measured using microstructure electrodes that penetrate the stratum corneum at multiple different frequencies;

FIG. 8A is a graph illustrating changes in bioimpedance gradients measured using microstructure electrodes that penetrate the stratum corneum following successive bouts of physical exertion;

FIG. 8B is a graph illustrating different impedance gradients measured using microstructure electrodes that penetrate the stratum corneum following successive bouts of physical exertion;

FIGS. 9A and 9B are graphs of example impedance measurements at 10 Hz and 100,000 Hz, respectively performed during repeated sequences of rest and exercise;

FIG. 10 is a schematic diagram of an example of a basic biophysical model;

FIG. 11A is a graph of an example of a large Electrode Polarization contribution that inhibits a major portion of the device response;

FIG. 11B is a graph of an example of a lower Electrode Polarization contribution that allows for greater frequency spectrum availability for observations of in-vivo effects;

FIG. 12A is a graph of example sensing device responses to different saline concentrations;

FIG. 12B is a graph of an example of an initial model fit up to the entire measurement which doesn't fit the high-frequency knee-feature;

FIG. 12C is a graph of an example of a revised model that provides a better fit for the entire measurement;

FIG. 13A is a graph of an example model impedance response for an uncoated microstructure device in-vitro;

FIG. 13B is a graph of an example model impedance response for a Parylene coated microstructure device in-vitro;

FIG. 13C is a graph of an example model impedance response for a partially coated microstructure device in-vitro;

FIG. 13D is a graph of an example model phase response for an uncoated microstructure device in-vitro depicting the alpha-dispersion;

FIG. 13E is a graph of an example model phase response for a Parylene coated microstructure device in-vitro, no alpha dispersion is visible;

FIG. 13F is a graph of an example model phase response for an etched microstructure device in-vitro, depicting a delayed alpha-dispersion;

FIG. 14A is a graph of an example in-vitro temperature response for an uncoated microstructure device;

FIG. 14B is a graph of an example model for in-vitro temperature response for an uncoated microstructure device;

FIG. 14C is a graph of an example of extracted solution resistance as effected by the change in temperature along with calculated temperature coefficients;

FIG. 15A is a graph of an example of in-vitro response of the microstructure device showing the expected impedance magnitude;

FIG. 15B is a graph of an example of in-vivo response of the microstructure device showing the expected impedance magnitude;

FIG. 15C is a graph of an example of in-vitro response of the microstructure device showing the expected phase response;

FIG. 15D is a graph of an example of in-vivo response of the microstructure device showing the expected phase response;

FIGS. 15E and 15F are schematic diagrams of a proposed model to exploit dispersions to interrogate intra-extra cellular response;

FIG. 16A is a graph of impedance frequency sweeps of surface electrodes on the skin, for dry skin, mild perspiration and heavy perspiration;

FIG. 16B is a graph of impedance frequency sweeps of microstructure electrodes within the skin, for dry skin, mild perspiration and heavy perspiration;

FIG. 17 is a graph of raw impedance measurements for an individual during a dehydration experiment;

FIGS. 18A and 18B are graphs of example magnitude and phase impedance measurements performed during heating and cooling;

FIGS. 19A and 19B are graphs of example magnitude and phase impedance measurements performed using an uncoated microstructure device;

FIGS. 19C and 19D are graphs of example magnitude and phase impedance measurements performed using a Parylene etched microstructure device; and,

FIGS. 19E and 19F are graphs of example magnitude and phase impedance measurements performed using surface impedance measurements.

### DETAILED DESCRIPTION OF THE PREFERRED EMBODIMENTS

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The terms “about” and “approximately” are used herein to refer to conditions (e.g. amounts, levels, concentrations, time, etc.) that vary by as much as 20% (i.e. ±20%), especially by as much as 10%, 9%, 8%, 7%, 6%, 5%, 4%, 3%, 2% or 1% to a specified condition.

As used herein, the term “and/or” refers to and encompasses any and all possible combinations of one or more of the associated listed items, as well as the lack of combinations when interpreted in the alternative (or).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “plurality” is used herein to refer to more than one, such as 2 to 1×10¹⁵ (or any integer therebetween) and upwards, including 2, 10, 100, 1000, 10000, 1×10⁶, 1×10⁷, 1×10⁸, 1×10⁹, 1×10¹⁰, 1×10¹¹, 1×10¹², 1×10¹³, 1×10¹⁴, 1×10¹⁵, etc. (and all integers therebetween).

The term “subject” as used herein refers to a vertebrate subject, particularly a mammalian subject, for whom monitoring and/or diagnosis of a disease, disorder or condition is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular, the subject is a human.

### System for Performing Measurements

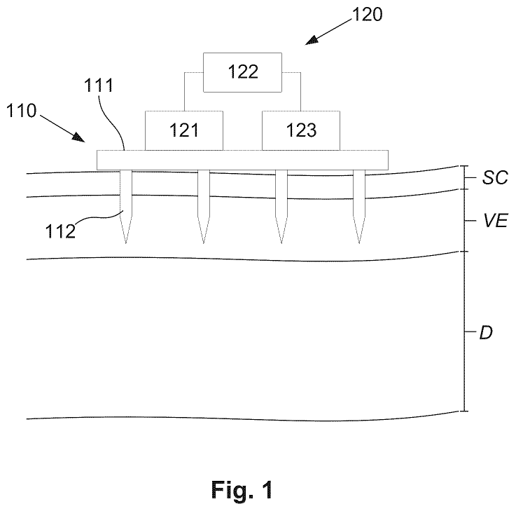

An example of a system for performing fluid level measurements on a biological subject will now be described with reference to FIG. 1.

In this example, the system 120 includes at least one substrate 111 having a plurality of microstructures 112. In use, the microstructures are configured to breach a functional barrier associated with a subject. In the current example, the functional barrier is the stratum corneum SC, and the microstructures are configured to breach the stratum corneum SC by penetrating the stratum corneum SC and entering at least the viable epidermis VE. In one particular example, the microstructures are configured to not penetrate a boundary between the viable epidermis VE and the dermis D, although this is not essential and structures that penetrate into the dermis could be used as will be described in more detail below.

The nature of the microstructure will vary depending upon the preferred implementation, but typically structures, such as plates, blades, or the like, are used, as will be described in more detail below, although this is not essential and other configurations, such as microneedles, could be used.

The substrate and microstructures could be manufactured from any suitable material, and the material used may depend on the intended application, for example depending on whether there is a requirement for the structures to be optically and/or electrically conductive, or the like. The substrate can form part of a patch 110, which can be applied to a subject, although other arrangements could be used for example, having the substrate form part of a housing containing other components.

At least some of the microstructures include an electrode, which could be formed by the body of the microstructure, so that the microstructure is the electrode, or which could be a surface electrode provided on the microstructure. At least one sensor 121 is provided, which is operatively connected to an electrode on at least one microstructure 112, thereby allowing response signals, and in particular electrical response signals, to be measured from respective microstructures 112. Additionally, at least one signal generator 123 is provided, which is operatively connected to an electrode on at least one microstructure 112, thereby allowing stimulatory signals, and in particular, electrical stimulatory signals to be applied to respective microstructures 112.

It will be noted that whilst the term response signal will be understood to encompass signals that are intrinsic within the subject, such as ECG (Electrocardiograph) signals, or the like, in the current example, the response signals are typically signals that are inferred as a result of the application of electrical currents, such as bioimpedance signals, or the like.

The nature of the sensor will vary depending on the preferred implementation and the nature of the sensing being performed, but typically the sensor senses electrical signals, in which case the sensor could be a voltage or current sensor, or the like. Similarly the signal generator is typically a current or voltage source, or the like.

The manner in which the sensor 121 and signal generator 123 are connected to the microstructure(s) 112 will also vary depending on the preferred implementation. In one example, this is achieved using electrical connections between the microstructure(s) 112 and the sensor 121 and/or signal generator 123. Connections could also include wireless connections, allowing the sensor and/or signal generator to be located remotely, for example allowing a smart phone or other device with NFC (Near Field Communication) capabilities to be used to interrogate the patch and perform measurements. Furthermore, connections could be provided as discrete elements, although in other examples, the substrate provides the connection, for example, if the substrate is made from a conductive plate which is then electrically connected to some or all of the microstructures. As a further alternative, the sensor could be embedded within or formed from part of the microstructure, in which connections may not be required.

The sensor 121 and/or signal generator 123 can be operatively connected to all of the microstructures 112, with connections being collective and/or independent. For example, one or more sensors and/or signal generators could be connected to different microstructures to allow different measured response signals to be measured from different groups of microstructures 112. However, this is not essential, and any suitable arrangement could be used.

These options allow a range of different types of sensing to be performed, but typically includes detecting the body's response to applied electrical signals, for example to measure bioimpedance, bioconductance, or biocapacitance, and the term bioimpedance will generally be understood to be of the complex mathematical form and thereby encompass all measurements of these types, including the real and reactive components of an impedance measurement.

The system further includes one or more electronic processing devices 122, which can form part of a measuring device, and/or could include electronic processing devices forming part of one or more processing systems, such as computer systems, servers, client devices, or the like as will be described in more detail below. In use, the processing devices 122 are adapted to receive signals from the sensor 121 and either store or process the signals. For ease of illustration the remaining description will refer generally to a processing device, but it will be appreciated that multiple processing devices could be used, with processing distributed between the devices as needed, and that reference to the singular encompasses the plural arrangement and vice versa.

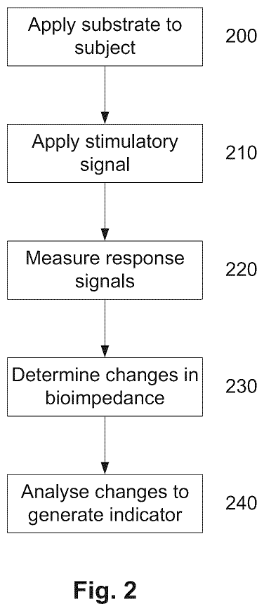

An example of the manner in which this is performed will now be described with reference to FIG. 2.

In particular, in this example, at step 200, the substrate is applied to the subject so that the one or more microstructures breach, and in one example, penetrate the functional barrier. In this example, the substrate is applied to skin, so that the microstructures penetrate the stratum corneum and enter the viable epidermis as shown in FIG. 1. This could be achieved manually and/or through the use of an actuator, to help ensure successful penetration.

At step 210, the signal generator is used to apply electrical stimulation to the electrodes, allowing response signals within the subject to be measured at step 220, with signals indicative of the measured response signals being provided to the electronic processing device 122.

The one or more processing devices then analyse multiple response signals measured over time to determine changes in bioimpedance at step 230, with the changes in bioimpedance being analysed to generate an indicator at step 240, which is typically at least partially indicative of a fluid status of the subject. For example, the indicator could be indicative of fluid levels, which are in turn indicative of hydration of the subject, or could be indicative of whether the subject is over, under, adequately hydrated, or undergoing restoration (restoring fluid levels between different compartments). Additionally and/or alternatively, the processing device could generate a recommendation for an intervention, for example recommending fluids are ingested to aid rehydration, or trigger an action, such as alerting a clinician, trainer or guardian, or the like.

The analysis can be performed in any suitable manner, and this will vary depending on nature of the measurements being performed. In one particular example, bioimpedance signals are used to calculate fluid levels, such as intra-cellular or extra-cellular fluid levels, and in particular changes, such as rates of change of intra-cellular and/or extra-cellular fluid levels, with these being used to calculate an indicator indicative of whether the subject is over-hydrated, under-hydrated, undergoing dehydration or undergoing restoration (returning to a normal hydration state), or the like. In this instance, measurements could be performed at particular frequencies indicative of intra or extracellular fluid levels, or alternatively measurements at multiple frequencies could be used to derive parameters indicative of intra or extracellular fluid levels.

In any event, it will be appreciated that the above described system operates by providing microstructures that are configured to breach the stratum corneum, allowing these to be used to apply stimulatory signals and measure response signals within the subject, and in particular, within the epidermis and/or dermis. These response signals can then be processed and subsequently analysed, allowing fluid levels to be derived, which could be indicative of specific measurements, hydration trends, or general hydration levels, or the like. In particular, in one preferred example, the system can be configured so that fluid level measurements are performed within the epidermis only, which in turn allows measures of body hydration to be performed with improved accuracy, providing higher quality data for more precise measures of body hydration. Furthermore, constraining the location in which measurements are performed ensures these are repeatable, allowing for more accurate longitudinal monitoring.

In contrast to traditional approaches, breaching and/or at least partially penetrating the stratum corneum allows measurements to be performed from within the epidermis and/or the dermis, which results in a significant improvement in the quality and magnitude of response signals that are detected. In particular, this ensures that the response signals accurately reflect conditions within the epidermis, such as the impedance of cells, tissue, interstitial fluid, or the like, as opposed to traditional external measurements, which are unduly influenced by the barrier properties, or the environment outside the barrier, such as the physical properties of the skin surface, such as the skin material properties, presence or absence of hair, sweat, mechanical movement of the applied sensor, or the like. Additionally, by penetrating the stratum corneum but not the dermis, this allows measurements to be constrained to the epidermis only, thereby avoiding interference from fluid level changes in the dermis.

For example, this allows accurate measurement of fluid levels within the body which would otherwise be unduly influenced by skin factors. For example, in the case of impedance measurements, microstructure electrodes tend to measure different parts of the equivalent circuit of human skin impedances as opposed to standard surface electrodes, which is indicative of the fact that the microstructure electrodes can selectively measure the impedance of the skin strata and do not measure whole skin or tissue impedance, meaning the measured impedance is more indicative of dynamic changes within the body. As the contribution of the skin surface and dermis impedance are significant in magnitude this can result in changes in impedance within the tissue being masked, meaning skin surface based measurements are less likely to be able to detect meaningful changes.

A further issue with skin based impedance measurements is that fields generated tend to pass through the stratum corneum and dermis, and are not constrained to the epidermis. Conversely, the above described minimally invasive patch allows electrical interrogation at precise, shallow skin layers using multi-frequency bioimpedance approaches. Interrogating shallowly removes the confounding effects of unknown tissue types such as bone, air and muscle. Discrimination of impedance contributions of intracellular fluid (ICF) and extracellular fluid (ECF) is possible on the basis of frequency. In contrast, current methods do not have the ability to discriminate between ICF and ECF which impairs the ability to both measure the temporal dynamics of fluid shifts and discriminate different classes of dehydration such as hypotonic, hypertonic or isotonic water loss. However, it will be appreciated that whilst the minimally invasive approach allows for impedance measurements to be constrained to the epidermis, this is not essential, and the approach could also be used to allow impedance measurements to be additionally and/or alternatively performed in the dermis, or other parts of the body.

Additionally, in some examples, the microstructures only penetrate the barrier a sufficient distance to allow a measurement to be made. For example, in the case of skin, the microstructures are typically configured to enter the viable epidermis and not enter the dermal layer. This results in a number of improvements over other invasive techniques, including avoiding issues associated with penetration of the dermis, such as pain caused by exposure of nerves, erythema, petechiae, or the like. Avoiding penetrating the dermal boundary also significantly reduces the risk of infection, allowing the microstructures to remain embedded for prolonged periods of time, such as several days, which in turn can be used to perform longitudinal monitoring over prolonged time periods.

It will be appreciated that the ability of the microstructures to remain in-situ is particularly beneficial, as this ensures that measurements are made at the same site within the subject, which reduces inherent variability arising from inaccuracies of replacement of measuring equipment which can arise using traditional techniques, whilst further allowing for substantially continuous monitoring. This allows changes in bioimpedance to be tracked more accurately, and in one particular example, tracked more accurately with respect to events that perturb fluid levels, such as commencing and/or ceasing physical exertion, taking medication, or the like. Despite this, it will be appreciated that the system can be used in other manners, for example to perform single time point monitoring, or the like.

Thus, the above arrangement can be provided as part of a wearable device, enabling measurements to be performed that are significantly better than existing surface based measurement techniques, for example by providing access to dynamic signals within the skin that cannot otherwise be measured through the stratum corneum, but whilst allowing measurements to be performed whilst the subject is undergoing normal activities and/or over a prolonged period of time. This in turn enables measurements to be captured that are more accurately reflective of the health or other status of the subject. For example, this allows variations in a subject's condition during a course of the day to be measured, during physical activities, and avoids measurements being made under artificial conditions, such as within a clinic, which are not typically indicative of the actual condition of the subject. This also allows monitoring to be performed substantially continuously, which can allow conditions to be detected as they arise, for example, in the case of myocardial infarction, cardiovascular disease, vomiting, diarrhoea or similar, which can allow more rapid intervention to be sought.

Further variations will become apparent from the following description.

In one example, the bioimpedance is measured at a single frequency, measured at multiple different frequencies and/or derived from impedance measurements performed at multiple different frequencies. For example, the system can use Bioimpedance Analysis (BIA) in which a single low frequency signal is injected into the subject S, with the measured impedance being used directly in the determination of biological parameters. In one example, the applied signal has a relatively low frequency, such as below 100 kHz, more typically below 50 kHz and more preferably below 10 kHz. In this instance, such low frequency signals can be used as an estimate of the impedance at zero applied frequency, which better characterise the electrical properties of extracellular fluid.

Alternatively, the applied signal can have a relatively high frequency, such as above 100 kHz, above 200 kHz, and more typically above 500 kHz, or 1000 kHz. In this instance, such high frequency signals can be used as an estimate of the impedance at infinite applied frequency, which is in turn indicative of a combination of the extracellular and intracellular fluid levels.

Alternatively and/or additionally, the system can use Bioimpedance Spectroscopy (BIS) in which impedance measurements are performed at multiple frequencies, which can then be used to derive information regarding both intracellular and extracellular fluid levels, for example by fitting measured impedance values to a Cole-Cole model.

In one example, the bioimpedance is indicative of one or more of intracellular fluid levels, extracellular fluid levels and blood/plasma fluid levels. Thus, in one example, the system uses a three compartment model, which includes intra and extra cellular fluids, and blood plasma, with the system examining changes in impedance resulting from movement of fluid between these different compartments in order to assess the fluid status of the subject, and thereby generate the indicator.

The change in bioimpedance could include any one or more of a change in a bioimpedance magnitude, a change in a bioimpedance phase angle, a change in intracellular fluid levels, a change in extracellular fluid levels and a change in blood fluid levels.

In one example, the changes are monitored relative to a baseline, so the system is configured to determine one or more baseline bioimpedance(s) and then analyse changes in bioimpedance(s) relative to the baseline bioimpedance(s). Thus, baseline bioimpedance(s) could be used to establish baseline extracellular and/or intracellular fluid levels, with subsequent measured bioimpedance(s) being used to establish changes extracellular and/or intracellular fluid levels relative to the baseline(s).

In one example, the processing device can be configured to determine a perturbation event, such as a change in a physical activity state of the subject, and then analyse the changes in bioimpedance at least in part in accordance with the perturbation event, for example measuring an impedance prior to a person undertaking a physical activity, with differences in impedance measured before and after the activity being used to monitor fluid status.

In one particular example, the processing device can be configured to determine a change in bioimpedance measured before and after the perturbation event, determine a change in bioimpedance measured during the perturbation event, determine a change in bioimpedance during a time period after the perturbation event and then determine a rate of change in bioimpedance during a time period after the perturbation event. Thus, this approach examines shifts in fluids, for example, between different compartments, after a perturbation event, for example when a subject is resting post physical exertion. In a further example, the processing device can compare multiple changes in bioimpedance, each change in bioimpedance being associated with a respective perturbation event and then determine the indicator based on the multiple changes in bioimpedance. For example, this could examine changes in impedance over multiple resting periods occurring between bouts of physical exertion. In one particular implementation of this approach, the processing device can determine a gradient of a rate of change in bioimpedance after each of multiple perturbation events and determine the indicator based on the changes in the gradients, for example based on whether the gradients are increasing or decreasing.

It will be appreciated that the above described approach could be performed for any perturbation event that influences a subject's fluid levels, including commencing or ending physical activity, performing ongoing physical activity, heating, cooling, changing posture, ingesting fluids, administration of medication, administration of a pharmacological agent, undergoing a medical procedure, such as dialysis, undergoing a physiological perturbation, administration of intravenous fluids, administration of intravenous blood, onset of illness or disease, or the like. In these examples, the processing device could determine the perturbation event based on one or more of user input commands, signals from at least one sensor, changes in a subject movement, changes in a subject posture, changes in a subject temperature, changes in a subject heart rate, and/or changes in a subject respiratory rate.

Thus, in one example, the system includes a sensor that is mounted on the substrate and/or provided within a housing attached to the substrate, allowing perturbation events to be detected, although this is not essential and alternatively sensing could be performed by analysing signals acquired from a separate device, such as an physical exertion tracker or similar.

The nature of the microstructures and the manner in which these are arranged will vary depending on the preferred implementation. For example, the microstructures could be arranged in pairs, with the bioimpedance being measured between multiple pairs of electrodes, optionally using pairs of electrodes with different spacings to thereby allow different measurements to be performed. For example, performing measurements with different spacings can target fluids at different depths within the body, which in turn can be useful in identifying in which compartments fluid is present. For example, measurements constrained to the viable epidermis will not typically capture fluid levels in blood plasma and instead will only include fluid levels from intra and extracellular fluids.

Similarly, the microstructures could be arranged in rows with the bioimpedance being measured between electrodes on different rows and optionally electrodes on different rows of microstructures with different spacings.

In one example, operation of the signal generator is controlled by the processing device, allowing the processing device to control the signal generator to thereby cause a measurement to be performed, for example by applying an electrical signal to allow an impedance measurement to be performed.

The signal generator and/or sensor can be connected to the microstructures via connections, including conductive connections, such as wires, or conductive tracks on a substrate, or could be formed by a conductive substrate. Connections could also include wireless connections, such as short-range radio frequency wireless connections, inductive connections, or the like. In one example, inductive connections can be used to transmit signals and power, so that for example, inductive coupling could be used to power electronic circuits mounted on the substrate. This could be used to allow basic processing to be performed onboard the substrate, such as amplifying and process impedance changes, using a simple integrated circuit or similar, without requiring an in-built power supply on the substrate.

In one example, the system can include response microstructures used to measure response signals and/or stimulation microstructures used to apply stimulation signals to the subject. Thus, stimulation and response could be measured via different microstructures, in which case the substrate typically incorporates response connections for allowing response signals to be measured and stimulation connections allowing stimulation signals to be applied. In some examples, multiple stimulation and response connections are provided, allowing different measurements to be performed via different connections. For example, different types of measurements could be performed via different microstructures or different parts of given microstructures, to enable multi-modal sensing. Additionally and/or alternatively, the same type of measurements could be performed at different locations and/or depths, for example to identify localised issues. In other cases, stimulation and measurement could be performed via the same connections, for example when making bipolar impedance measurements.

Signals could be applied to or measured from individual microstructures and/or to different parts of microstructures, which can be useful to discern features at different locations and/or depths within the body, for example to measure fluid levels within different compartments. Additionally, and/or alternatively, signals could be applied to or measured from multiple microstructures collectively, which can be used to improve signal quality, or perform measurements, such as bipolar, tetra-polar, or other multi-polar impedance measurements using multiple microstructures.

In one particular example, sensors and/or signal generators can be connected to microstructures via one or more switching devices, such as multiplexers, allowing signals to be selectively communicated between the sensor or signal generator and different microstructures. The processing device is typically configured to control the switches, allowing a variety of different sensing and stimulation to be achieved under control of the processing device. In one example, this allows at least some electrodes to be used independently of at least some other electrodes. This ability to selectively interrogate different electrodes can provide benefits.

For example, this allows measurements to be performed via different electrodes to allow for spatial discrimination and hence mapping to be performed. For example, interrogating electrodes at different locations on a patch enables a map of measurements at different depths within tissue to be constructed.

In one example, as described in more detail below, when electrodes are provided as pairs, this allows some pairs of electrodes to be used independently of other pairs. In one particular example, electrodes and/or pairs of electrodes, can be arranged in rows, and this can allow measurements to be performed on a row by row basis, although this is not essential and other groupings could be used.

The nature of the substrate and/or microstructures will vary depending upon the preferred implementation. The substrate and microstructures could be made from similar and/or dissimilar materials, and could be integrally formed, or made separately and bonded together. In preferred examples, the substrate and microstructures are formed from a polymer or similar. Microstructures can also be provided on one or more substrates, so for example, signals could be measured or applied between microstructures on separate substrates.

It will be appreciated that the particular material used will depend on the intended application, so for example different materials will be used if the microstructure needs to be conductive as opposed to insulative. Insulating materials, such as polymers and plastics could be doped so as to provide required conductivity, for example via doping with micro or nano sized metal particles, or conductive composite polymers. If doping is used, this could involve using graphite or graphite derivates, including 2D materials such as graphene and carbon nanotubes, with these materials also being useable as stand-alone materials or as dopants in blends with polymers or plastics.

The substrate and microstructures can be manufactured using any suitable technique. For example, in the case of silicon-based structures, this could be performed using etching techniques. Polymer or plastic structures could be manufactured using additive manufacturing, such as 3D printing, moulding, imprinting, imprint lithography, stamping, hot embossing, or the like.

In one example, the substrate could be at least partially flexible in order to allow the substrate to conform to the shape of a subject and thereby ensure penetration of the microstructures into the viable epidermis, or other functional barrier. In this example, the substrate could potentially be a polymer such as PET (Polyethylene Terephthalate), a textile or fabric, with electrodes and circuitry woven in, or multiple substrates could be mounted on a flexible backing, to provide a segmented substrate arrangement. Alternatively, the substrate could be shaped to conform to a shape of the subject, so that the substrate is rigid but nevertheless ensures penetration of the microstructures.

The microstructures could have a range of different shapes and could include ridges or needles, although plates or blades, or similar, are typically preferred. In this regard, the terms plates and blades are used interchangeably to refer to microstructures having a width that is of a similar order of magnitude (or larger) in size to the length, but which are significantly (such as an order of magnitude) thinner. Such arrangements are particularly beneficial as these can support larger surface area electrodes, thereby maximising the effective electrode surface area for a given number of microstructures.

The microstructures can be tapered to facilitate insertion into the subject, and can have different shapes, for example depending on the intended use. The microstructures typically have a rounded rectangular shape when viewed in cross section through a plane extending laterally through the microstructures and parallel to but offset from the substrate. The microstructures may include shape changes along a length of the microstructure. For example, microstructures could include a shoulder that is configured to abut against the stratum corneum to control a depth of penetration and/or a shaft extending to the tip, with the shaft being configured to control a position of the tip in the subject and/or provide a surface for an electrode.

Microstructures can have a rough or smooth surface, or may include surface features, such as pores, raised portions, serrations, or the like, which can increase surface area and/or assist in penetrating or engaging tissue, to thereby anchor the microstructures within the subject. This can also assist in reducing biofouling, for example by prohibiting the adherence and hence build-up of biofilms. The microstructures might also be hollow or porous and can include an internal structure, such as holes or similar, in which case the cross sectional shape could also be at least partially hollow. In particular embodiments, the microstructures are porous, which may increase the effective surface area of the microstructure. The pores may be of any suitable size to allow an analyte of interest to enter the pores, but exclude one or more other analytes or substances, and thus, will depend on the size of the analyte of interest. In some embodiments, the pores may be less than about 10 μm in diameter, preferably less than about 1 μm in diameter.

Different microstructures could be provided on a common substrate, for example providing different shapes of microstructure to achieve different functions. In one example, this could include performing different types of measurement. In other examples, microstructures could be provided on different substrates, for example, allowing sensing to be performed via microstructures on different patches, for example, performing whole of body impedance measurements between patches provided at different locations on a subject.

In a further example, at least part of the substrate could be coated with an adhesive coating in order to allow the substrate and hence patch, to adhere to the subject.

As previously mentioned, when applied to skin, the microstructures typically enter the viable epidermis and preferably do not enter the dermis. But this is not essential, and for some applications, it may be necessary for the microstructures to enter the dermis, for example projecting shortly through the viable epidermis/dermis boundary or entering into the dermis a significant distance, largely depending on the nature of the sensing being performed. In one example, for skin, the microstructures have a length that is at least one of less than 2500 μm, less than 1000 μm, less than 750 μm, less than 600 μm, less than 500 μm, less than 400 μm, less than 300 μm, less than 250 μm, greater than 100 μm, greater than 50 μm and greater than 10 μm, but it will be appreciated that other lengths could be used. More generally, when applied to a functional barrier, the microstructures typically have a length greater than the thickness of the functional barrier, at least 10% greater than the thickness of the functional barrier, at least 20% greater than the thickness of the functional barrier, at least 50% greater than the thickness of the functional barrier, at least 75% greater than the thickness of the functional barrier and at least 100% greater than the thickness of the functional barrier.

In another example, the microstructures have a length that is no more than 2000% greater than the thickness of the functional barrier, no more than 1000% greater than the thickness of the functional barrier, no more than 500% greater than the thickness of the functional barrier, no more than 100% greater than the thickness of the functional barrier, no more than 75% greater than the thickness of the functional barrier or no more than 50% greater than the thickness of the functional barrier. This can avoid deep penetration of underlying layers within the body, which can in turn be undesirable, and it will be appreciated that the length of the microstructures used will vary depending on the intended use, and in particular the nature of the barrier to be breached, and/or signals to be applied or measured. The length of the microstructures can also be uneven, for example, allowing a blade to be taller at one end than another, which can facilitate penetration of the subject or functional barrier.

Similarly, the microstructures can have different widths depending on the preferred implementation. Typically, the widths are at least one of less than 25% of the length, less than 20% of the length, less than 15% of the length, less than 10% of the length, or less than 5% of the length. Thus, for example, when applied to the skin, the microstructures could have a width of less than 50 μm, less than 40 μm, less than 30 μm, less than 20 μm or less than 10 μm. However, alternatively, the microstructures could include blades, and could be wider than the length of the microstructures. In some examples, the microstructures could have a width of less than 2500 μm, less than 1000 μm, less than 500 μm or less than 100 μm. In blade microstructure examples, it is also feasible to use microstructures having a width substantially up to the width of the substrate.

In general the thickness of the microstructures is significantly lower in order to facilitate penetration and is typically less than 1000 μm, less than 500 μm, less than 200 μm, less than 100 μm, less than 50 μm, less than 20 μm, less than 10 μm, at least 1 μm, at least 0.5 μm or at least 0.1 μm. In general the thickness of the microstructure is governed by mechanical requirements, and in particular the need to ensure the microstructure does not break, fracture or deform upon penetration. However, this issue can be mitigated through the use of a coating that adds additional mechanical strength to the microstructures.

In one specific example, for epidermal sensing, the microstructures have a length that is less than 300 μm, greater than 50 μm, greater than 100 μm and about 200 μm, and, a width that is greater than or about equal to a length of the microstructure, and is typically less than 300 μm, greater than 50 μm and about 150 μm. In another example, for dermal sensing, the microstructures have a length that is less than 450 μm, greater than 100 μm, and about 250 μm, and, a width that is greater than or about equal to a length of the microstructure, and at least of a similar order of magnitude to the length, and is typically less than 450 μm, greater than 100 μm, and about 250 μm. In other examples, longer microstructures could be used, so for example for hyperdermal sensing, the microstructures would be of a greater length. The microstructures typically have a thickness that is less than the width, significantly less than the width and of an order of magnitude smaller than the width. In one example, the thickness is less than 50 μm, greater than 10 μm, and about 25 μm, whilst the microstructure typically includes a flared base for additional strength, and hence includes a base thickness proximate the substrate that is about three times the thickness, and typically is less than 150 μm, greater than 30 μm and about 75 μm. The microstructures typically have a tip that has a length less than 50% of a length of the microstructure, at least 10% of a length of the microstructure and more typically about 30% of a length of the microstructure. The tip further has a sharpness that is at least 0.1 μm, less than 5 μm and typically about 1 μm.

In one example, the microstructures have a relatively low density, such as less than 10,000 per cm², such as less than 1000 per cm², less than 500 per cm², less than 100 per cm², less than 10 per cm² or even less than 5 per cm². The use of a relatively low density facilitates penetration of the microstructures through the stratum corneum and in particular avoids the issues associated with penetration of the skin by high density arrays, which in turn can lead to the need for high powered actuators in order for the arrays to be correctly applied. However, this is not essential, and higher density microstructure arrangements could be used, including less than 50,000 microstructures per cm², less than 30,000 microstructures per cm², or the like. As a result, the microstructures typically have a spacing that is less than 20 mm, less than 10 mm, less than 1 mm, less than 0.1 mm or less than 10 μm.

In one specific example, the microstructures have a density that is less than 100 per cm², greater than 10 per cm², and about 30 per cm², leading to a spacing of less than 2 mm, more than 10 μm, and about 1.0 mm, 0.5 mm, 0.2 mm or 0.1 mm.

It should be noted that in some circumstances, microstructures are arranged in pairs, with the microstructures in each pair having a small spacing, such as less than 10 μm, whilst the pairs have a great spacing, such as more than 1 mm, in order to ensure a low overall density is maintained. However, it will be appreciated that this is not essential, and higher densities could be used in some circumstances.

As mentioned above, at least some of microstructures include an electrode, which can be used to apply electrical signals to a subject, measure intrinsic or extrinsic response electrical signals, for example measuring ECG or impedances. The microstructures could be made from a metal or other conductive material, so that the entire microstructure constitutes the electrode, or alternatively the electrode could be coated or deposited onto the microstructure, for example by depositing a layer of gold to form the electrode. The electrode material could include any one or more of gold, silver, colloidal silver, colloidal gold, colloidal carbon, carbon nano materials, platinum, titanium, stainless steel, or other metals, or any other biocompatible conductive material.

In a further example, the microstructure could include an electrically conductive core or layer covered by a non-conductive layer (insulating), with openings providing access to the core to allow conduction of electrical signals through the openings, to thereby define electrodes. In one example, the insulating layer extends over part of a surface of the microstructure, including a proximal end of the microstructure adjacent the substrate. The insulating layer could extend over at least half of a length of the microstructure and/or about 90 μm of a proximal end of the microstructure, and optionally, at least part of a tip portion of the microstructure. In one specific example, this is performed so the non-insulating portion is provided in the epidermis, so stimulatory signals are applied to and/or response signals received from, the epidermis.

The insulating layer could also extend over some or all of a surface of the substrate. In this regard, in some examples connections are formed on a surface of the substrate, in which case a coating, and in particular a dielectric coating such as Parylene, could be used to isolate these from the subject. For example, electrical tracks on a surface of the substrate could be used to provide electrical connections to the electrodes, with an insulating layer being provided on top of the connections to ensure the connections do not make electrical contact with the skin of the subject, which could in turn adversely affect measured response signals. For example, this prevents electrical contact with the skin surface, in turn preventing surface moisture, such as sweat, from influencing the measurements.

In one example, the microstructures include plates having a substantially planar face having an electrode thereon. The use of a plate shape maximizes the surface area of the electrode, whilst minimizing the cross sectional area of the microstructure, to thereby assist with penetration of the microstructure into the subject. This also allows the electrode to act as a capacitive plate, allowing capacitive sensing to be performed. In one example, the electrodes have a surface area of at least at least 10 mm², at least 1 mm², at least 100,000 μm², 10,000 μm², at least 7,500 μm², at least 5,000 μm², at least 2,000 μm², at least 1,000 μm², at least 500 μm², at least 100 μm², or at least 10 μm². In one example, the electrodes have a width or height that is up to 2500 μm, at least 500 μm, at least 200 μm, at least 100 μm, at least 75 μm, at least 50 μm, at least 20 μm, at least 10 μm or at least 1 μm. In the case of electrodes provided on blades, the electrode width could be less than 50000 μm, less than 40000 μm, less than 30000 μm, less than 20000 μm, less than 10000 μm, or less than 1000 μm, as well as including widths outlined previously. In this regard, it will be noted that these dimensions apply to individual electrodes, and in some examples each microstructure might include multiple electrodes.

In one specific example, the electrodes have a surface area of less than 200,000 μm², at least 2,000 μm² and about 22,500 μm², with the electrodes extending over a length of a distal portion of the microstructure, optionally spaced from the tip, and optionally positioned proximate a distal end of the microstructure, again proximate the tip of the microstructure. The electrode can extend over at least 25% and less than 50% of a length of the microstructure, so that the electrode typically extends over about 60 μm of the microstructure and hence is positioned in a viable epidermis of the subject in use. Other lengths, such as 90 μm or 150 μm could be used for dermal sensing.

In one example, at least some of the microstructures are arranged in groups, such as pairs and/or rows, with response signals or stimulation being measured from or applied to the microstructures within the group. The microstructures within the group can have a specific configuration to allow particular measurements to be performed. For example, when arranged in pairs or rows, a separation distance between microstructures in the pair or the different rows can be used to influence the nature of measurements performed. For example, when performing bioimpedance measurements, if the separation between the microstructures is greater than a few millimetres, this will tend to measure properties of interstitial fluid located between the electrodes, whereas if the distance between the microstructures is reduced, measurements will be more influenced by microstructure surface properties, such as the presence of materials bound to the surface of the microstructures. Measurements are also influenced by the nature of the applied stimulation, so that for example, current at low frequencies will tend to flow though extra-cellular fluids, whereas current at higher frequencies is more influenced by intra-cellular fluids.

In one particular example, plate microstructures are provided in pairs, with each pair including spaced apart plate microstructures having substantially planar electrodes in opposition. This can be used to generate a highly uniform field in the subject in a region between the electrodes, and/or to perform capacitive or conductivity sensing of substances between the electrodes. However, this is not essential, and other configurations, such as circumferentially spacing a plurality of electrodes around a central electrode, can be used. Typically the spacing between the electrodes in each group is typically less than 50 mm, less than 20 mm, less than 10 mm, less than 1 mm, less than 0.1 mm or less than 10 μm, although it will be appreciated that greater spacings could be used, including spacing up to dimensions of the substrate and/or greater, if microstructures are distributed across multiple substrates.

Thus, in one specific example, at least some of the microstructures are arranged in pairs or rows, with response signals being measured between microstructures in the pair or different rows and/or stimulation being applied between microstructures in the pair or different rows. Each pair of microstructures typically includes spaced apart plate microstructures having substantially planar electrodes in opposition and/or spaced apart substantially parallel plate microstructures, and similar arrangements could be used for rows of microstructures, with microstructures on different rows having substantially planar electrodes in opposition and/or spaced apart substantially parallel plate microstructures.

In one example, at least some microstructures are angularly offset, and in one particular example, are orthogonally arranged. Thus, in the case of plate microstructures, at least some pairs of microstructures extend in different and optionally orthogonal directions. This distributes stresses associated with insertion of the patch in different directions, and also acts to reduce sideways slippage of the patch by ensuring plates at least partially face a direction of any lateral force. Reducing slippage either during or post insertion helps reduce discomfort, erythema, or the like, and can assist in making the patch comfortable to wear for prolonged periods. Additionally, this can also help to account for any electrical anisotropy within the tissue, for example as a result of fibrin structures within the skin, cellular anisotropy, or the like.

In one specific example, adjacent pairs of microstructures are angularly offset, and/or orthogonally arranged, and additionally and/or alternatively, pairs of microstructures can be arranged in rows, with the pairs of microstructures in one row are orthogonally arranged or angularly offset relative to pairs of microstructures in other rows.

In one specific example, when pairs of microstructures are used, a spacing between the microstructures in each pair is typically less than 0.25 mm, more than 10 μm and about 0.1 mm, whilst a spacing between groups of microstructures is typically less than 1 mm, more than 0.2 mm and about 0.5 mm. Such an arrangement helps ensure electrical signals are primarily applied and measured within a pair and reduces cross talk between pairs, allowing independent measurements to be recorded for each pair of microstructures/electrodes.

Additionally, the microstructures can incorporate one or more materials or other additives, either within the body of the microstructure, or through addition of a coating containing the additive. The nature of the additive will vary depending on the preferred implementation and could include a material to reduce biofouling, a material to attract at least one substance to the microstructures, or a material to repel at least one substance from the microstructures. Example materials include polyethylene, polyethylene glycol, polyethylene oxide, zwitterions, peptides, hydrogels and SAMs.

The material can be contained within the microstructures themselves, for example by impregnating the microstructures during manufacture, or could be provided in a coating. For example, in the case of moulded patches manufactured using a polymer material, the material can be introduced into the mould together with the polymer material so that the material is distributed throughout the structures. In this example, the polymer can be arranged so that pores form within the structures during the curing process.

It will be appreciated that microstructures could be differentially coated, for example by coating different microstructures with different coatings, and/or by coating different parts of the microstructures with different coatings.

The nature of the coating and the manner in which this is applied will vary depending on the preferred implementation and techniques such as dip coating, spray coating, jet coating or the like, could be used, as described above. The thickness of the coating will also vary depending on the circumstances and the intended functionality provided by the coating. For example, if the coating is used to provide mechanical strength, or contains a payload material to be delivered to the subject, a thicker coating could be used, whereas if the coating is used for sensing other applications, a thinner coating might be required. In one particular example, coatings can be used to selectively insulate part of the surface of the microstructures, so that a conductive microstructure is insulated outside of the body, preventing impedance measurements being adversely affected by surface moisture, such as sweat.

In one example, the system includes a housing containing at least the sensor, the signal generator and one or more electronic processing devices, and optionally including other components, such as an actuator, power supply, wireless transceiver, or the like. In one particular example, the housing provides reader functionality that can be used to interrogate the microstructures, and which can be provided in an integrated device, or could be provided remote to the substrate and engaged or provided in proximity with the substrate when readings are to be performed.

In the integrated configuration, the reader is typically mechanically connected/integrated with the patch during normal use, allowing measurements to be performed automatically. For example, continual monitoring could be performed, with a reading being performed every 1 second to daily or weekly typically every 2 to 60 minutes, and more typically every 5 to 10 minutes. The timing of readings can vary depending on the nature of the measurement being performed and the particular circumstance. So for example, an athlete might wish to undergo more frequent monitoring while competing in an event, and then less frequent monitoring during post event recovery. Similarly, for a person undergoing medical monitoring, the frequency of monitoring may vary depending on the nature and/or severity of a condition. In one example, the frequency of monitoring can be selected based on user inputs and/or could be based on a defined user profile, or the like.

In the integrated arrangement, the reader can be connected to the patch using conventional resistance bridge circuitry, with analogue to digital conversion being used to perform measurements.

Alternatively, the reader can be separate, which allows the reader to be removed when not in use, allowing the user to wear a patch without any integrated electronics, making this less intrusive. This is particularly useful for applications, such as sports, geriatric and paediatric medicine, or the like, where the presence of a bulkier device could impact on activities. In this situation, the reader is typically brought into contact or proximity with the patch allowing readings to be performed on demand. It will be appreciated that this requires a user/person to drive the interrogation. However, the reader could include alert functionality to encourage interrogation.

Readings could be performed wirelessly, optionally using inductive coupling to both power the patch and perform the reading as will be described in more detail below, although alternatively, direct physical contact could alternatively be used. In this example, the microstructures and tissue form part of a resonant circuit with discrete inductance or capacitance, allowing the frequency to be used to determine the impedance and hence fluid levels. Additionally, and/or alternatively, ohmic contacts could be used, where the reader makes electrical contact with connectors on the patch.

In either case, some analysis and interpretation of the hydration signal may be performed in the reader, optionally allowing an indicator to be displayed on the reader using an output, such as an LED indicator, LCD screen, or the like. Additionally, and/or alternatively, audible alarms may be provided, for example providing an indication in the event that the subject is under or over hydrated. The reader can also incorporate wireless connectivity, such as Bluetooth, Wi-Fi or similar, allowing reading events to be triggered remotely and/or to allow data, such as impedance values, hydration indicators, or the like to be transmitted to remote devices, such as a client device, computer system, or cloud based computing arrangement.

In one example, the housing selectively couples to the substrate, allowing the housing and substrate to be attached and detached as needed. In one example, this could be achieved utilising any appropriate mechanism, such as electromagnetic coupling, mechanical coupling, adhesive coupling, magnetic coupling, or the like. This allows the housing and in particular sensing equipment to only be connected to the substrate as needed. Thus, a substrate could be applied to and secured to a subject, with a sensing system only being attached to the substrate as measurements are to be performed. However, it will be appreciated that this is not essential, and alternatively the housing and substrate could be collectively secured to the subject for example using an adhesive patch, adhesive coating on the patch/substrate, strap, anchor microstructures, or the like. In a further example, the substrate could form part of the housing, so that the substrate and microstructures are integrated into the housing.

When the housing is configured to attach to the substrate, the housing typically includes connectors that operatively connect to substrate connectors on the substrate, to thereby communicate signals between the signal generator and/or sensor, and the microstructures. The nature of the connectors and connections will vary depending upon the preferred implementation and the nature of the signal, and could include conductive contact surfaces that engage corresponding surfaces on the substrate, or could include wireless connections, such as tuned inductive coils, wireless communication antennas, or the like.

In one example, the system is configured to perform repeated measurements over a time period, such as a few hours, days, weeks, or similar. To achieve this, the microstructures can be configured to remain in the subject during the time period, or alternatively could be removed when measurements are not being performed. In one example, the actuator can be configured to trigger insertion of the microstructures into the skin and also allow for removal of the microstructures once the measurements have been performed. The microstructures can then be inserted and retracted as needed, to enable measurements to be performed over a prolonged period of time, without ongoing penetration of the skin. However, this is not essential and alternatively short term measurements can be performed, in which case the time period can be less than 0.01 seconds, less than 0.1 seconds, less than 1 second or less than 10 seconds. It will be appreciated that other intermediate time frames could also be used.

In one example, once measurements have been performed, the one or more electronic processing devices analyse the measured response signals to determine the indicator.

In one example, this is achieved by deriving at least one metric, which can then be used to determine an indicator. For example, the system could be configured to perform impedance measurements, with the metric corresponding to an impedance parameter, such as an impedance at a particular frequency, a phase angle, a temporal change, or similar. The metric can then be used to derive an indication of fluid levels, such as extra or intra cellular fluid levels, which can be used in generating the indicator.

In one example, the system can include a transmitter that transmits measured subject data, metrics or measurement data such as response signals or values derived from measured response signals, allowing these to be analysed remotely.

In one particular example, the system includes a wearable patch including the substrate and microstructures, and a monitoring device (also referred to as a “reader”) that performs the measurements. The monitoring device could be attached or integrally formed with the patch, for example mounting any required electronics on a rear side of the substrate. Alternatively, the reader could be brought into contact with the patch when a reading is to be performed. In either case, connections between the monitoring device could be conductive contacts, but alternatively could be indicative couplings, allowing the patch to be wirelessly interrogated and/or powered by the reader.

The monitoring device can be configured to cause a measurement to be performed and/or to at least partially analyse measurements. The monitoring device can control stimulation applied to at least one microstructure, for example by controlling the signal generator and/or switches as needed. This allows the monitoring device to selectively interrogate different microstructures, allowing different measurements to be performed, and/or allowing measurements to be performed at different locations.

The monitoring device could also be used to generate an output, such as an output indicative of the indicator or a recommendation based on the indicator and/or cause an action to be performed. Thus, the monitoring device could be configured to generate an output including a notification or an alert. This can be used to trigger an intervention, for example, indicating to a user that action is required. This could simply be an indication of an issue, such as telling a user they are dehydrated and/or could include a recommendation, such as telling the user to rehydrate, or seek medical attention or similar. The output could additionally and/or alternatively, include an indication of an indicator, such as a measured value, or information derived from an indicator. Thus, a hydration level could be presented to the user.

The output could be used to alert a caregiver that an intervention is required, for example transferring a notification to a client device and/or computer of the caregiver. In another example, this could also be used to control remote equipment. For example, this could be used to trigger a drug delivery system, such as an electronically controlled syringe injection pump, allowing an intervention to be triggered automatically. In a further example, a semi-automated system could be used, for example providing a clinician with a notification including an indicator, and a recommended intervention, allowing the clinician to approve the intervention, which is then performed automatically.

In one example, the monitoring device is configured to interface with a separate processing system, such as a client device and/or computer system. In this example, this allows processing and analysis tasks to be distributed between the monitoring device and the client device and/or computer system. For example, the monitoring device could perform partial processing of measured response signals, such as filtering and/or digitising these, providing an indication of the processed signals to a remote process system for analysis. In one example, this is achieved by generating subject data including the processed response signals, and transferring this to a client device and/or computer system for analysis. Thus, this allows the monitoring device to communicate with a computer system that generates, analyses or stores subject data derived from the measurement data. This can then be used to generate an indicator at least partially indicative of a health status associated with the subject.

It will also be appreciated that this allows additional functionality to be implemented, including transferring notifications to clinicians, or other caregivers, and also allowing for remote storage of data and/or indicators. In one example, this allows recorded measurements and other information, such as derived indicators, details of applied stimulation or therapy and/or details of other resulting actions, to be directly incorporated into an electronic record, such as an electronic medical record.

In one example, this allows the system to provide the data that will underpin the growing telehealth sector empowering telehealth systems with high fidelity and accurate clinical data to enable remote clinicians to gain the information they require, and they will be highly valued both in central hospitals and in rural areas away from centralized laboratories and regional hospitals. With time to treatment a strong predictor of improved clinical outcomes with heart attack patients, decentralized populations cannot rely solely on access to conventional large-scale hospitals. Accordingly, the system can provide a low cost, robust and accurate monitoring system, capable for example of diagnosing a heart attack, and yet being provided at any local health facility and as simple as applying a patch device. In this example, resources could be dispatched quickly for patients who test positive to troponin I, with no delay for cardiac troponin laboratory blood-tests. Similarly patients determined to be low-risk could be released earlier and with fewer invasive tests, or funneled into other streams via their GP etc.

In a further example, a client device such as a smart phone, tablet, or the like, is used to receive measurement data from the wearable monitoring device, generate subject data and then transfer this to the processing system, with the processing system returning an indicator, which can then be displayed on the client device and/or monitoring device, depending on the preferred implementation.

However, this is not essential and it will be appreciated that some or all of the steps of analysing measurements, generating an indicator and/or displaying a representation of the indicator could be performed on board the monitoring device. Again, it will be appreciated that similar outputs could also be provided to or by a remote processing system or client device, for example, alerting a clinician or trainer that a subject or athlete requires attention.

The reader could be configured to perform measurements automatically when integrated into or permanently/semi permanently attached to the patch, or could perform measurements when brought into contact with the patch if the reader is separate. In this latter example, the reader can be inductively coupled to the patch.

Thus, it will be appreciated that functionality, such as processing measured response signals, analysing results, generating outputs, controlling measurement procedures and/or therapy delivery could be performed by an on-board monitoring device, and/or could be performed by remote computer systems, and that the particular distribution of tasks and resulting functionality can vary depending on the preferred implementation.

In one example, the system includes a substrate coil positioned on the substrate and operatively coupled electronics, which are then connected to one or more microstructure electrodes, which could include microstructures that are electrodes, or microstructures including electrodes thereon. An excitation and receiving coil is provided, typically in a housing of a measuring device, such as an NFC enabled mobile phone, or other similar device with the excitation and receiving coil being positioned in proximity to the substrate coil in use. This is performed to inductively couple the excitation and receiving coil to the substrate coils, so that when an excitation signal is applied to the drive coil, this powers the electronics on the substrate, allowing a measurement to be performed, and results communicated back to the measuring device via the receiving coil.

Accordingly, it will be appreciated that this allows the wearable sensors to be passive if they harvest energy from external sources, or active if the energy to feed the electronics is obtained from a battery. The inclusion of energy harvesting capabilities allows for passive sensors with low costs or lifetimes extended beyond battery limitations.

The inclusion of energy harvesting into NFC chips allows for battery-less NFC sensor technology, the energy harvested from the radiofrequency (RF) interrogating signal from a reading device. This is particularly advantageous as this allows existing devices equipped with NFC capabilities to be used as a reader. However, it will be appreciated that there are several frequency bands for the application, including low frequency (LF), high frequency (HF), ultrahigh frequency (UHF), or microwave bands, and so reference to NFC should not be considered limiting.

It is also noted that at LF or HF a list is established by near-field communications (NFC) because the read range is less than the wavelength. Therefore, communication between the loop antennas of the reader and sensor is produced by inductive coupling. The limited read range offers advantage to improve privacy and device security under undesired access to information on the device. However, the distance over which reading can be performed is limited. If a larger communication range is required, UHF readers can be used, and whilst these are typically more expensive than those for NFC, the read range can be increased to reach several metres or more. UHF communication is based on the modulation of the far fields and the read range is higher than those based on near-field communications.

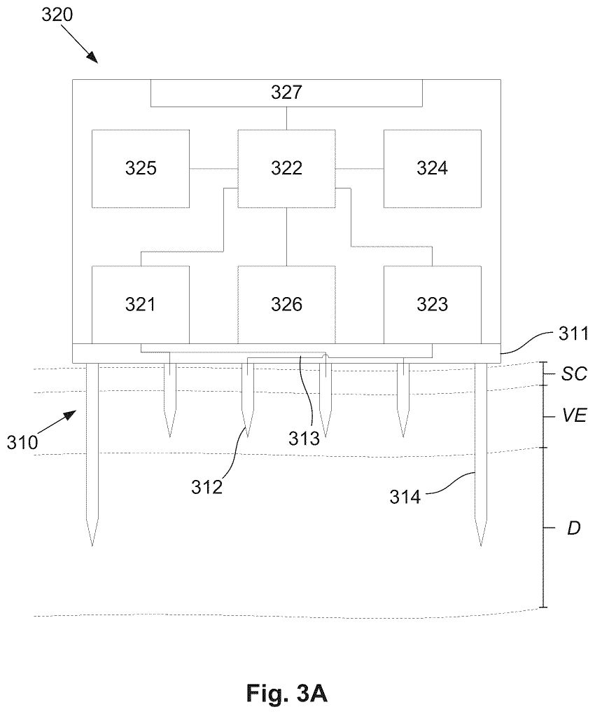

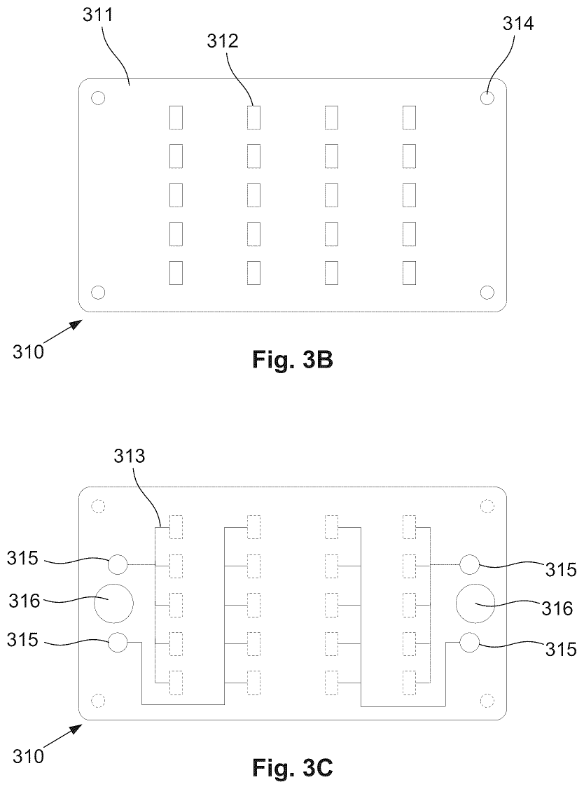

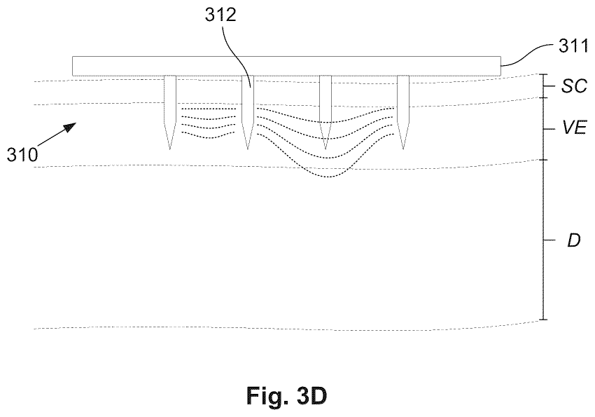

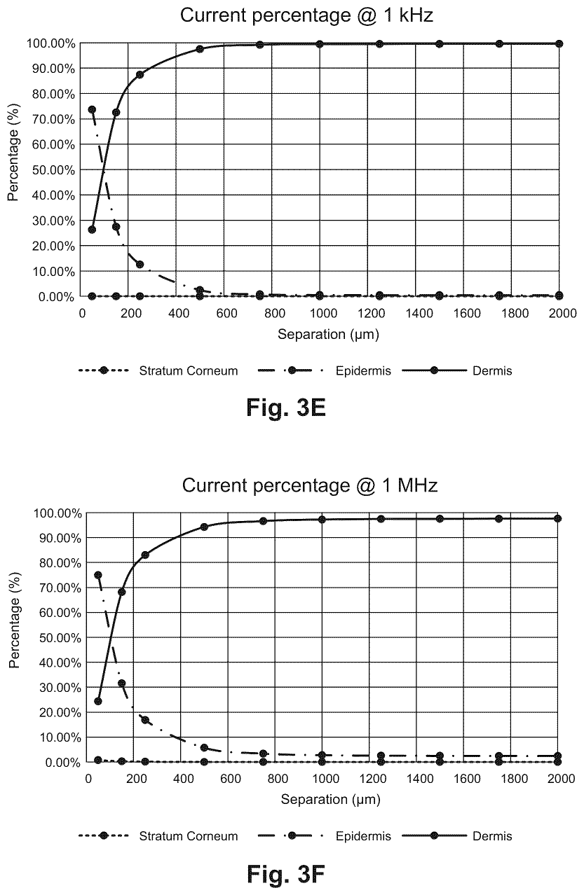

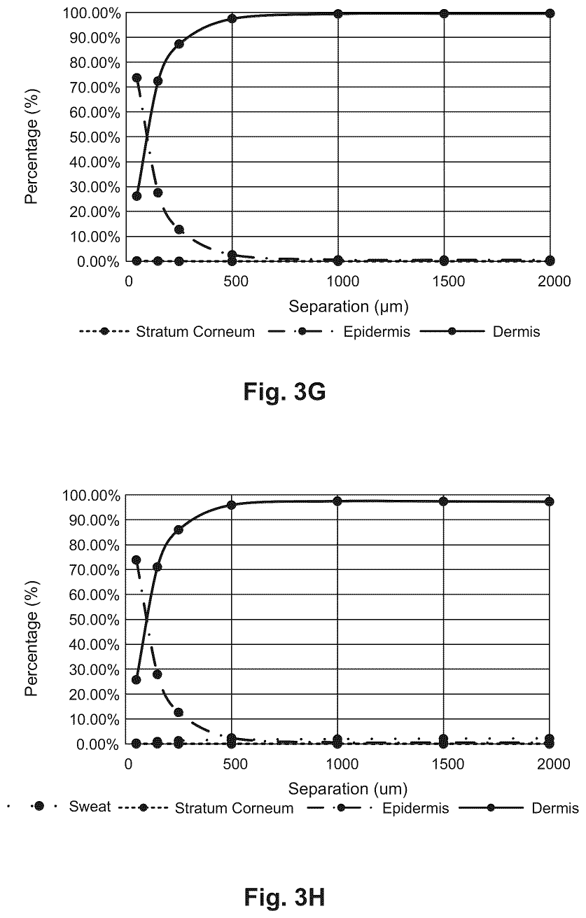

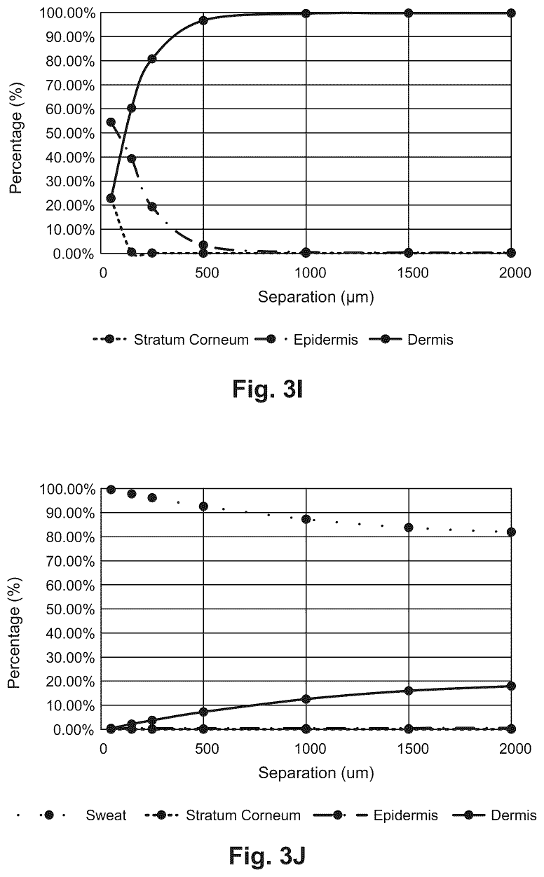

A further example of a system for performing measurements in the biological subject will now be described with reference to FIGS. 3A to 3C.

In this example, the system includes a monitoring device 320, including a sensor 321 and one or more electronic processing devices 322. The system further includes a signal generator 323, a memory 324, an external interface 325, such as a wireless transceiver, an actuator 326, and an input/output device 327, such as a touchscreen or display and input buttons, connected to the electronic processing device 322. These components are typically provided in a housing.

The nature of the signal generator 323 and sensor 321 will depend on the measurements being performed, and could include a current source and voltage sensor, laser or other electromagnetic radiation source, such as an LED and photodiode or CCD sensor, or the like. The actuator 326 is typically a spring or electromagnetic actuator in combination with a piezoelectric actuator or vibratory motor coupled to the housing, to bias and vibrate the substrate relative to an underside of the housing, to thereby urge the microstructures into the skin, whilst the transceiver is typically a short-range wireless transceiver, such as a Bluetooth system on a chip (SoC).

The processing device 322 executes software instructions stored in the memory 324 to allow various processes to be performed, including controlling the signal generator 323, receiving and interpreting signals from the sensor 321, generating measurement data and transmitting this to a client device or other processing system via the transceiver 325. Accordingly, the electronic processing device is typically a microprocessor, microcontroller, microchip processor, logic gate configuration, firmware optionally associated with implementing logic such as an FPGA (Field Programmable Gate Array), or any other electronic device, system or arrangement.

In use the monitoring device 320 is coupled to a patch 310, including a substrate 311 and microstructures 312, which are coupled to the sensor 321 and/or signal generator 323 via connections 313. The connections could include physical conductive connections, such as conductive tracks, although this is not essential and alternatively wireless connections could be provided, such inductive coupling or radio frequency wireless connections. In this example, the patch further includes anchor microstructures 314 that are configured to penetrate into the dermis and thereby assist in securing the patch to the subject.

An example of the patch 310 is shown in more detail in FIGS. 3B and 3C. In particular, in this example the substrate 311 is generally rectangular, with round corners to avoid discomfort when the substrate is applied to the subject's skin. The substrate 311 includes anchor microstructures 314 are provided proximate corners of the substrate 311 to help secure the substrate, whilst measurement microstructures 312 are arranged in an array or rows on the substrate. In this example, the array has a regular grid formation, with the microstructures 312 being in provided in equally spaced rows and columns, but this is not essential and alternative spacing configurations could be used, as will be described in more detail below.

In the example of FIGS. 3B and 3C, four connectors 315 are provided which are connected to respective microstructures 312 via connections 313 to allow stimulation signals and response signals to be applied to and measured from two sets of respective microstructures. This can be used to allow for symmetric or differential application and detection of signals, as opposed to asymmetric or single-ended application or detection, which is typically performed relative to a ground reference, and which is in turn generally noisier. However, it will be appreciated that for some detection modalities, such as bipolar impedance measurements, or the like, this is not relevant and single connections 313 may be provided.

In the example of FIGS. 3B and 3C, rows of microstructures are provided with measurements being performed between different rows, such as adjacent rows having a closer spacing or non-adjacent rows having a relatively large spacing, which can be used to enable different properties to be detected, or different forms of stimulation to be performed. For example, a greater electrode spacing will typically lead to electrical signals penetrating more deeply, allowing measurements to be performed into the dermis, which means that measured response signals can be indicative of fluid levels in blood plasma as well as intra and extra cellular fluids, as shown in FIG. 3D.

To test this, modelling was used to study electrical current density at different depths using different blade and microstructure arrangements, including two blade microstructures with a respective electrode with separations of 50, 150, 250, 500, 1000, 1500 and 2000 μm and two surface electrodes with separations of 50, 150, 250, 500, 1000, 1500 and 2000 μm.

FIGS. 3E and 3F demonstrate that for two blade microstructures with respective electrodes, the separation of the microstructures heavily influences the depth of penetration of the electrical field, with the field being constrained to the epidermis at lower separations and extending into the dermis for greater separations. Furthermore, this effect is more pronounced at lower frequencies, so that at lower frequencies the ability to measure fluid levels in the epidermis reduces at a lower separation than for higher frequencies.

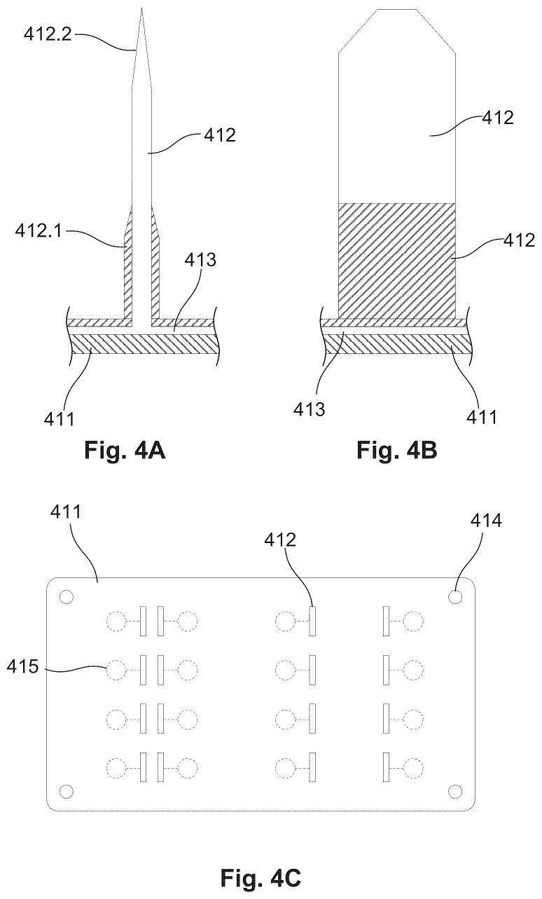

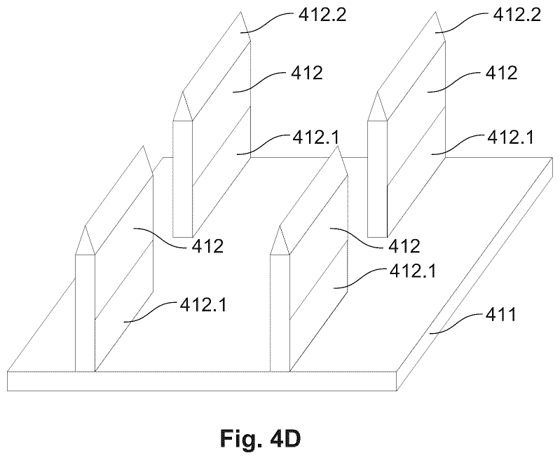

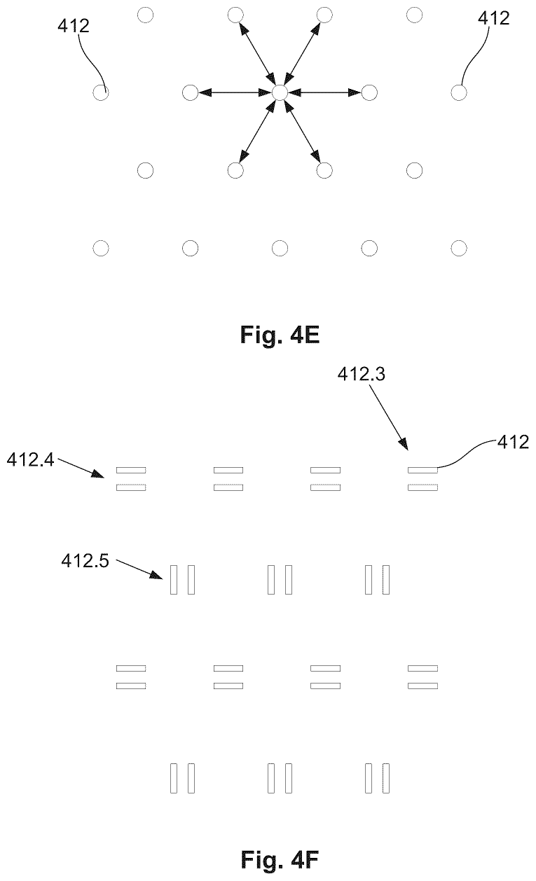

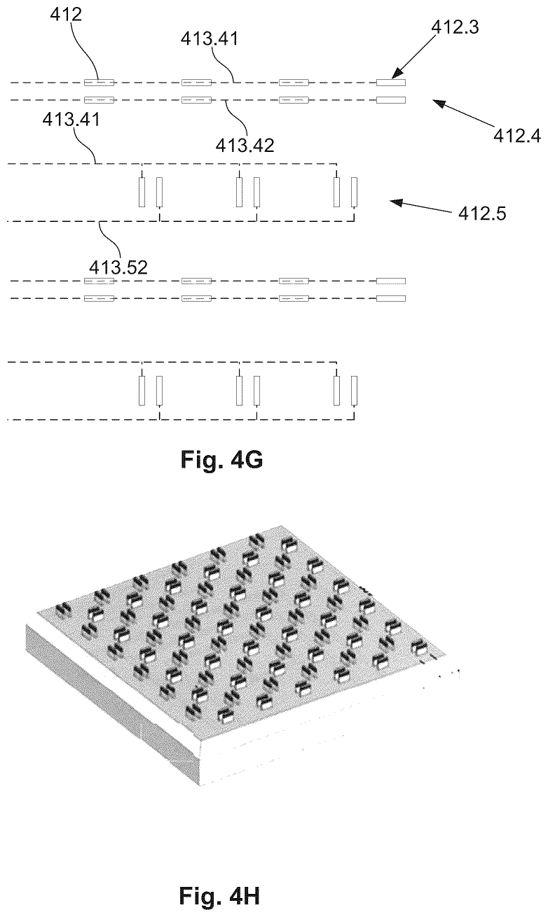

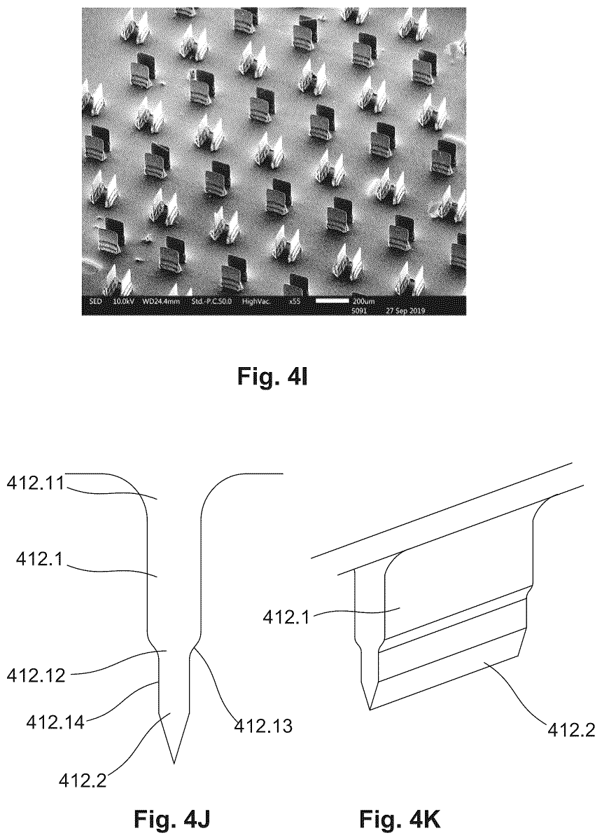

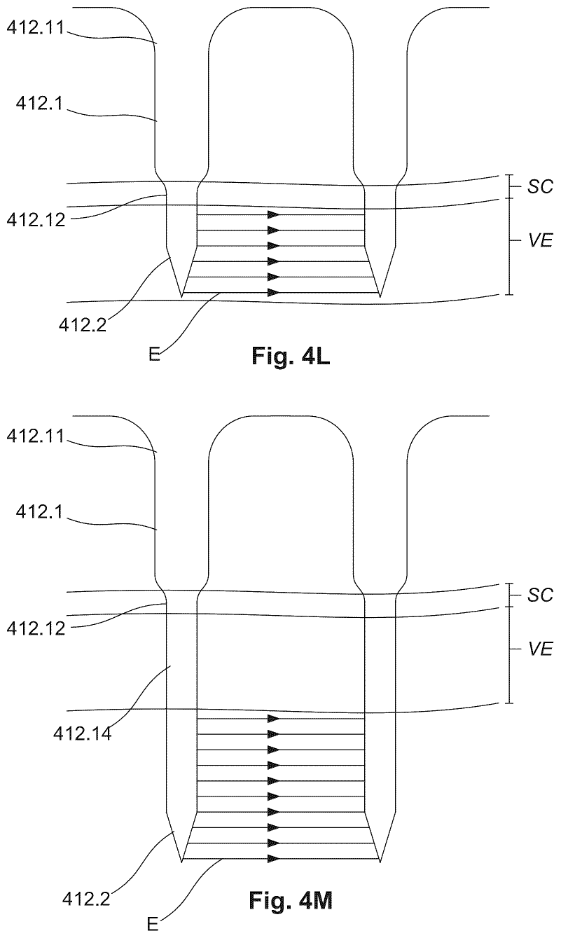

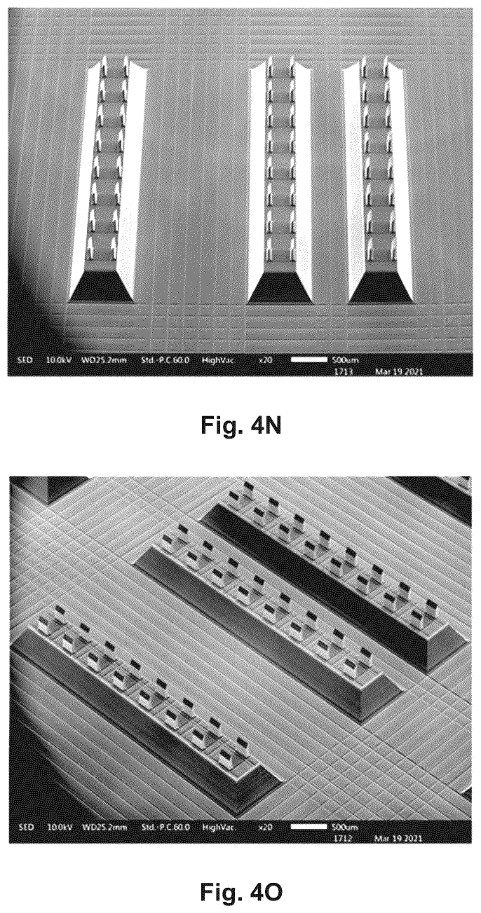

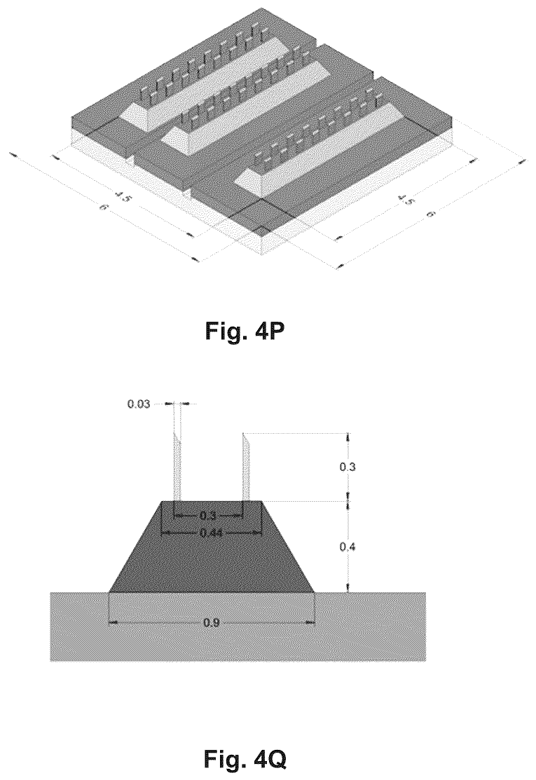

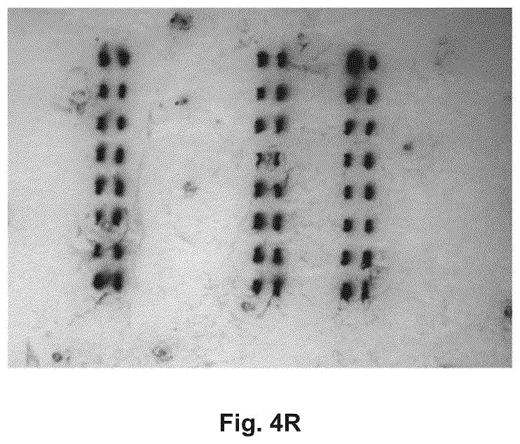

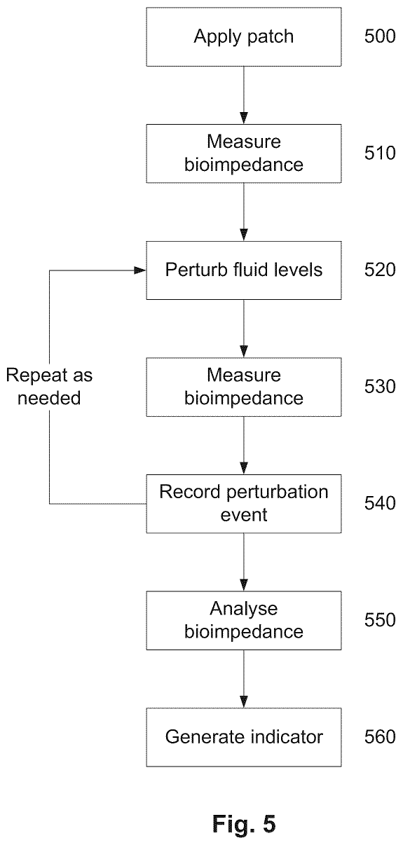

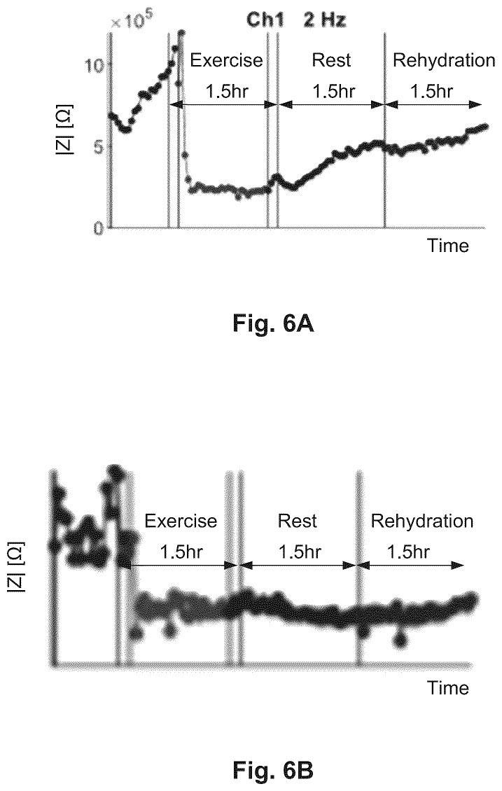

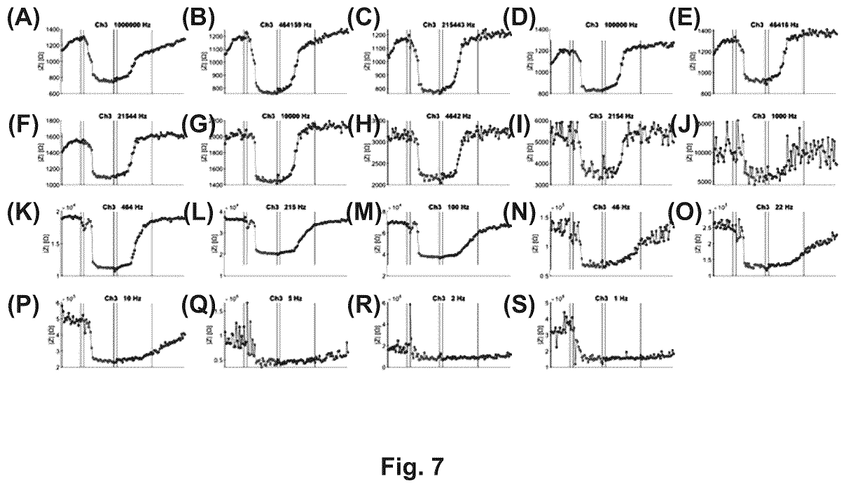

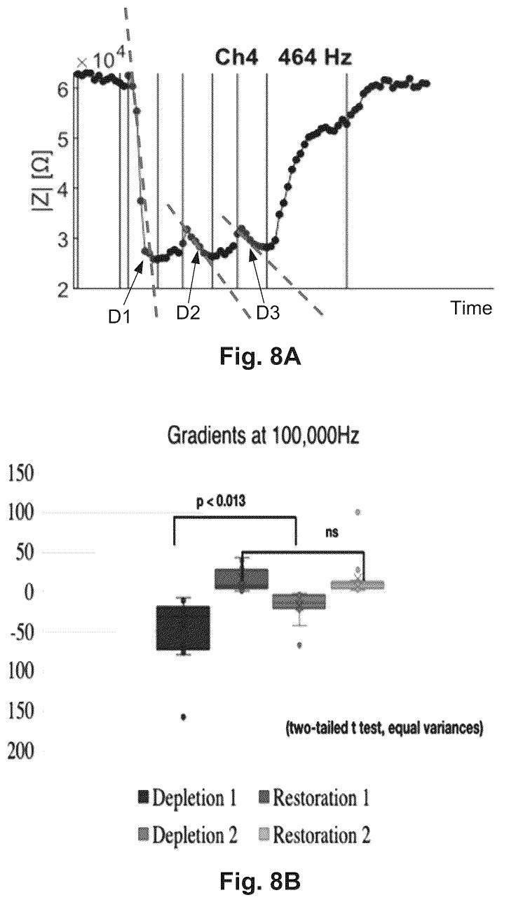

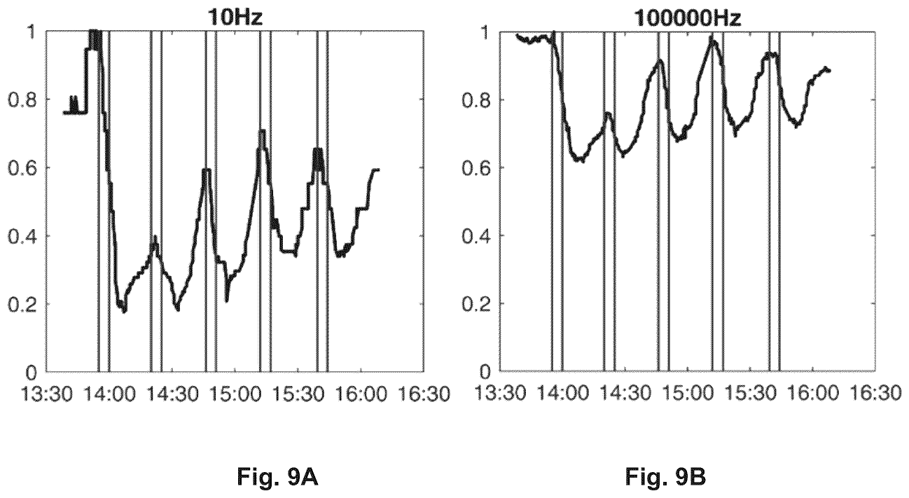

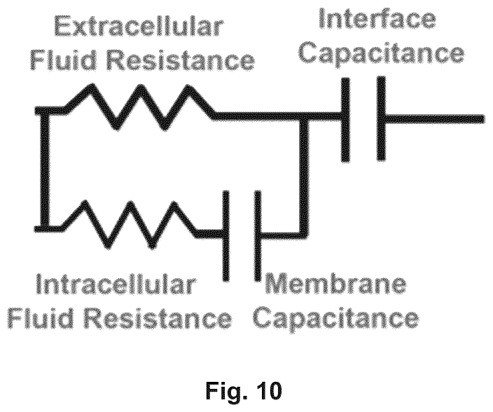

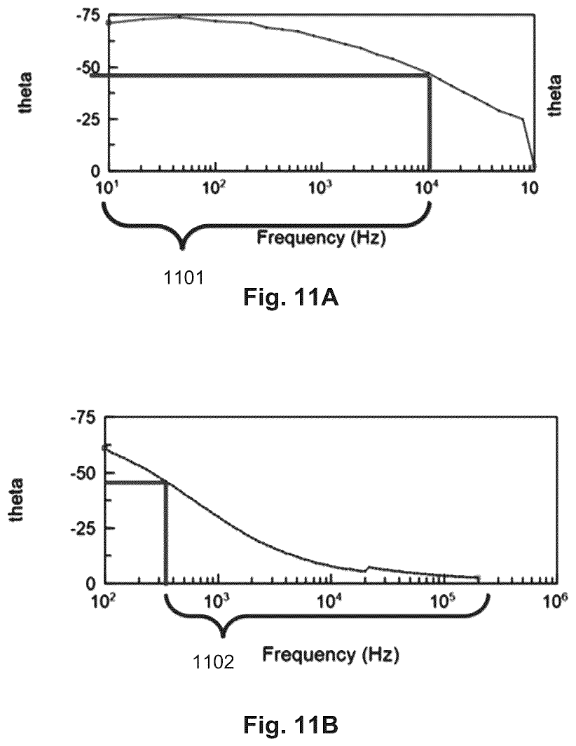

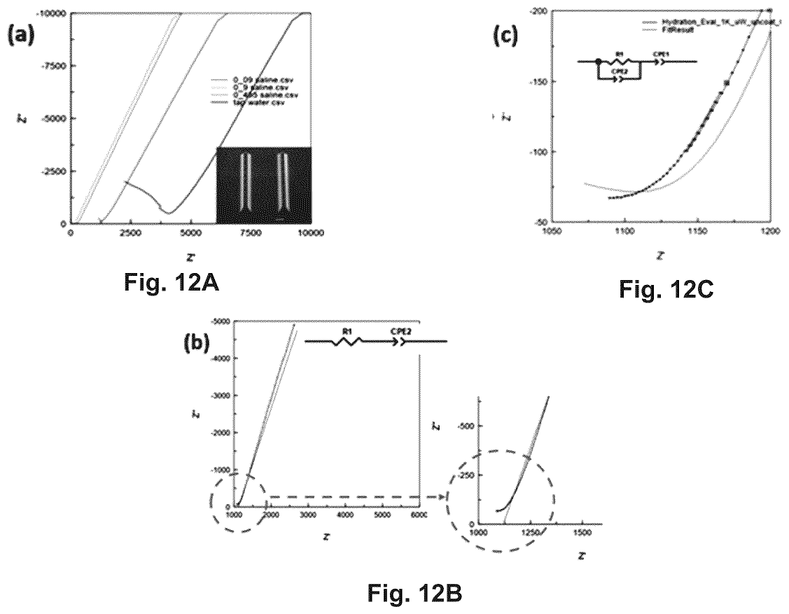

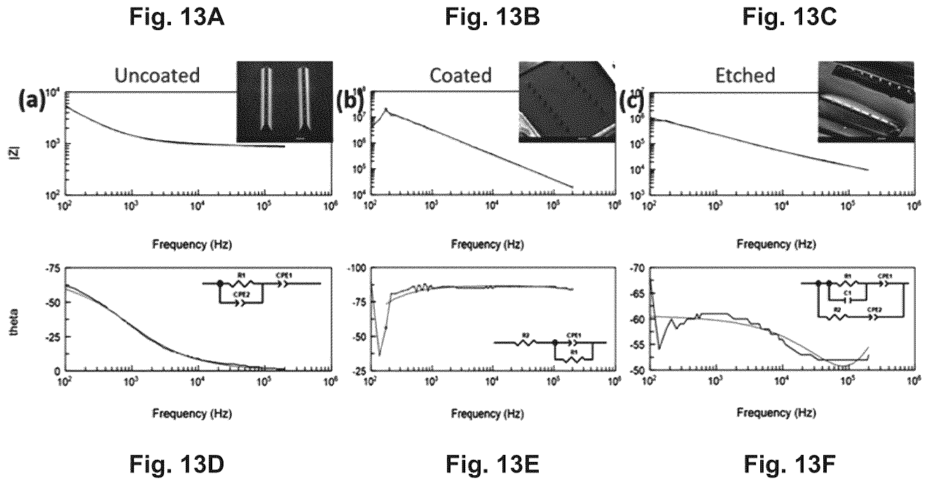

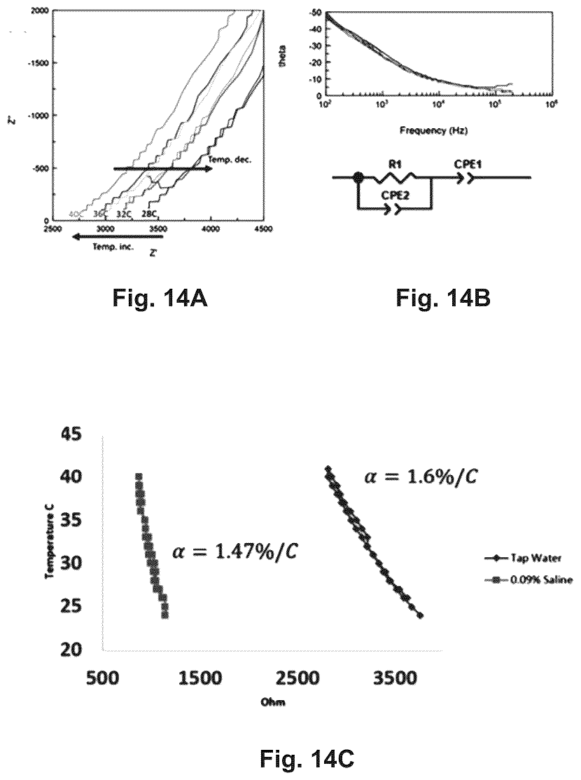

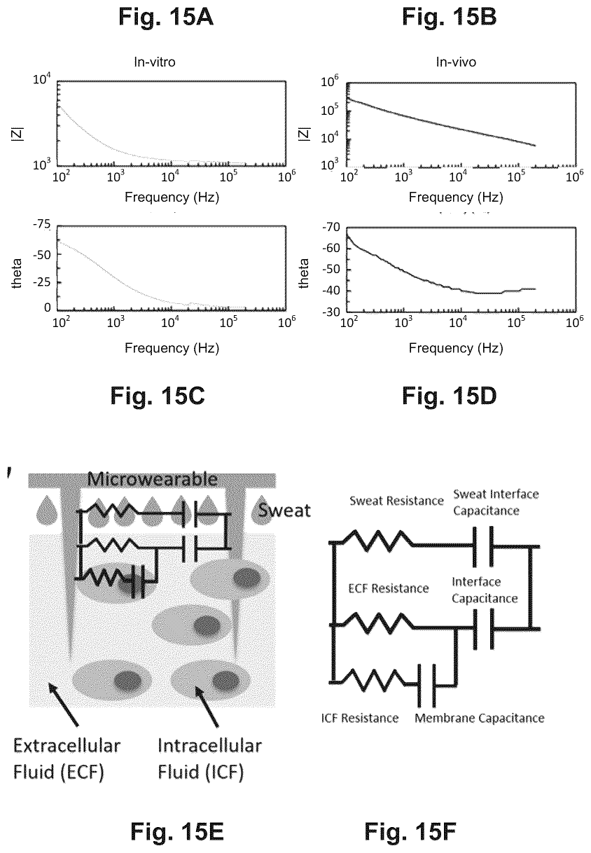

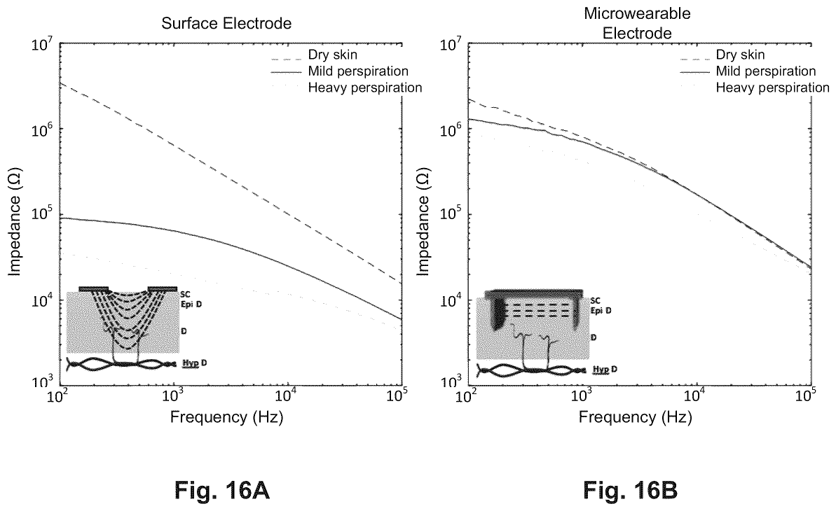

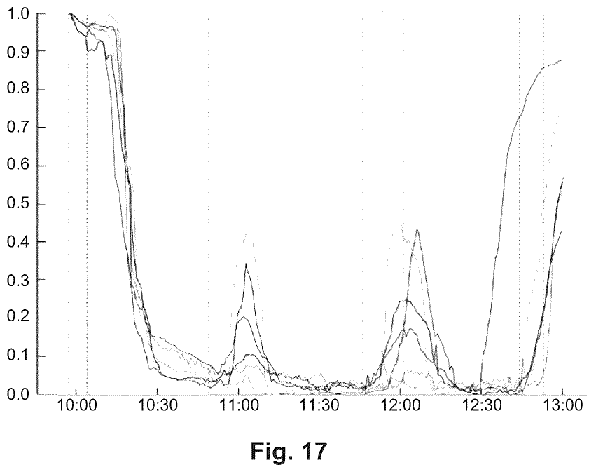

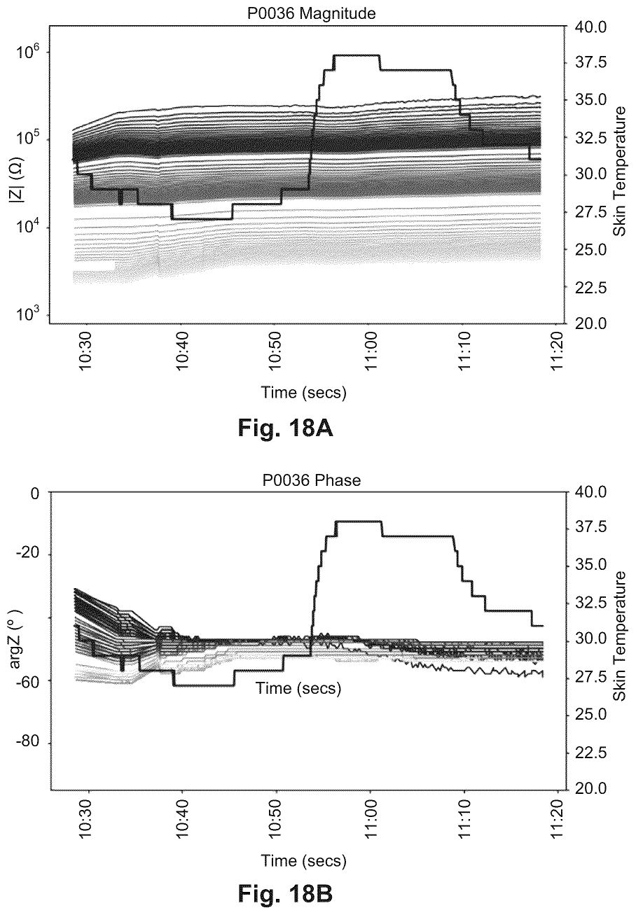

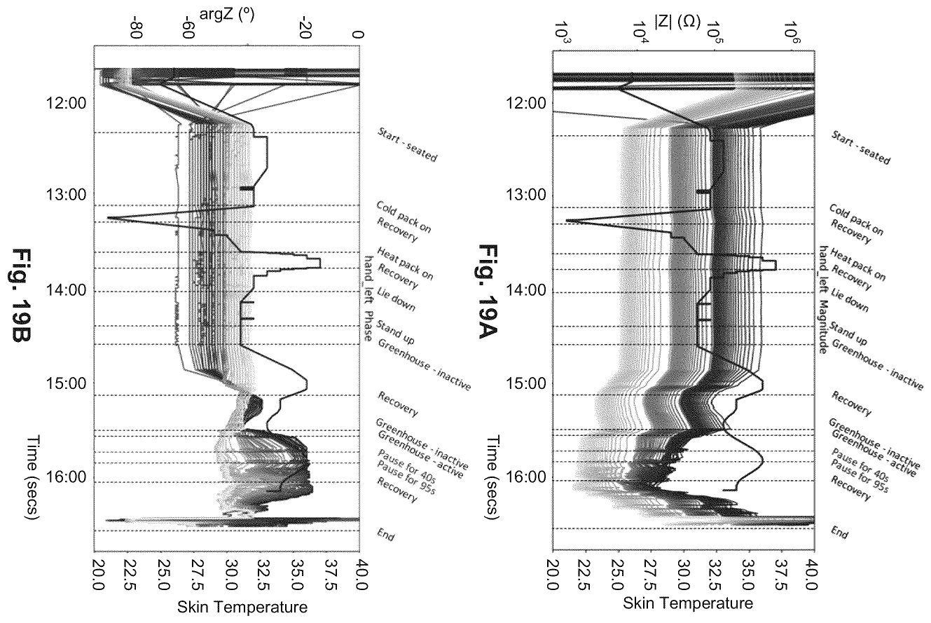

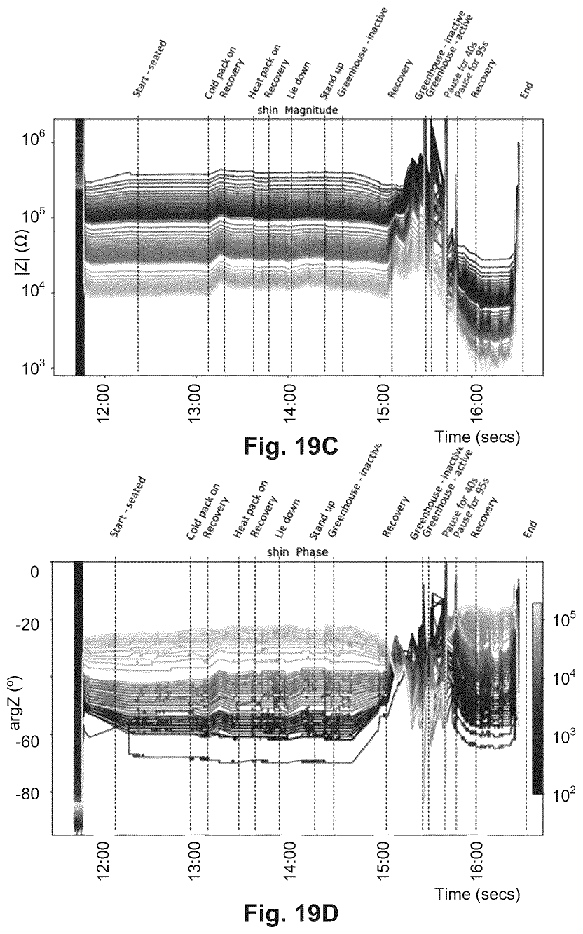

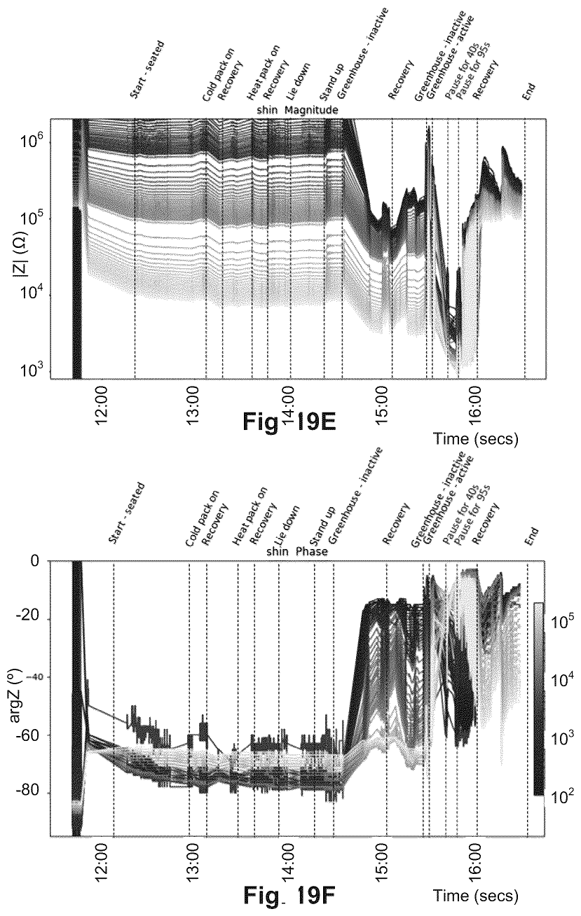

FIGS. 3G and 3H show results for two blade microstructures with respective electrodes in the absence and presence of sweat, whilst equivalent measurements for surface electrodes are shown in FIGS. 31 and 3J, respectively. These highlight that whilst blade microstructure electrode measurements are largely independent of sweat levels, the surface based impedance measurements are heavily dependent on sweat levels, to the extent that sweat largely swamps the measurements of fluid levels in the epidermis and to a lesser extent the dermis.

A specific example of a plate microstructure is shown is shown in FIGS. 4A to 4C.

In this example, the microstructure is a plate having a body 412 and a tip 412.2, which is tapered to facilitate penetration of the microstructure body 412 into the stratum corneum. In this example, the microstructure includes a polymer body 412 extending from a polymer substrate 411. The microstructure and upper surface of the substrate are typically coated with a conductive coating (not shown), so that the microstructure is conductive and in electrical contact with a connection 413 on a surface of the substrate, formed by the conductive coating. The substrate 411, the connection 413, and a lower part of the body 412 are covered by an insulating layer 412.1, such as a polymer, Parylene, or other material. In this instance, the insulating layer 412.1 covers the base of the microstructure 412 and the substrate 411 and connections 413, so that electrical signals are only communicated with tissue within the viable epidermis, thereby preventing surface moisture, such as sweat, interfering with measurements performed.

As shown in FIGS. 4C and 4D, different arrangements could be used but in general, pairs of microstructures are formed with the microstructures facing each other allowing signals to be applied between the microstructures or measured between the microstructures. Again, different separations between electrodes in pairs of electrodes can be used to allow different measurements to be performed and/or to alter the profile of stimulation of the tissue between the electrodes.

In the example shown, the blade tip is parallel to the substrate, but this is not essential and other configurations could be used, such as having a sloped tip, so that the blade penetrates progressively along the length of the blade as it is inserted, which can in turn facilitate penetration. The tip may also include serrations, or similar, to further enhance penetration.

As mentioned above, in one example, microstructures are provided in a regular grid arrangement. However, in another example, the microstructures are provided in a hexagonal grid arrangement as shown in FIG. 4E. This is particularly advantageous as each microstructure is equally spaced to all of the nearest neighbour microstructures, as shown by the arrows, meaning measurements can be performed relative to any adjacent microstructure without requiring response or stimulation signals to be modified to account for different spacings.

A further example arrangement is shown in FIGS. 4F to 4I, in which microstructures 412 are arranged in pairs 412.3, and with pairs arranged in offset rows, 412.4, 412.5. In this example, pairs in different rows are arranged orthogonally, so that the microstructures extend in different directions. This avoids all microstructures being aligned, which can render a patch vulnerable to lateral slippage in a direction aligned with the microstructures. Additionally arranging the pairs orthogonally reduces interference, such as cross talk, between different pairs of electrodes, improving measurement accuracy and accounting for tissue anisotropy, particularly when measurements are being performed via multiple microstructure pairs simultaneously.

In one example, pairs of microstructures in each row can be provided with respective connections 413.41, 413.42; 413.51, 413.52, allowing an entire row of microstructure pairs to be interrogated and/or stimulated simultaneously, whilst allowing different rows to be interrogated and/or stimulated independently.

A Scanning Electron Microscopy (SEM) image showing an array of pairs of offset plate microstructures is shown in FIG. 4I.

Specific examples of microstructures for performing measurements in the epidermis are shown in FIGS. 4J and 4K.

In this example, the microstructures are plates or blades, having a body 412.1, with a flared base 412.11, where the body joins the substrate, to enhance the strength of the microstructure. The body narrows at a waist 412.12 to define shoulders 412.13 and then extends to a tapered tip 412.2, in this example, via an untapered shaft 412.14. Typical dimensions are shown in Table 1 below.

TABLE 1

| Parameter | Min. | Typical | Max. | Units |
|---|---|---|---|---|
| Length | 50 | 150 | 300 | microns |
| Width | 50 | 150 | 300 | microns |
| Thickness | 10 | 40 | 80 | microns |
| Density | 10 | 30 | 200 | cm⁻² |
| Tip radius | 0.1 | 2 | 10 | microns |
| Surface area | 2,000 | 22,500 | 200,000 | micron² |
| per electrode | | | | |
| Buttress width | 30 | 75 | 150 | microns |
| at base | | | | |

An example of a pair of the microstructures on insertion into a subject is shown in FIGS. 4L and 4M.

In this example, the microstructures are configured so that the tip 412.2 penetrates the stratum corneum SC and enters the viable epidermis VE. The waist 412.12, and in particular the shoulders 412.13 abut the stratum corneum SC so that the microstructure does not penetrate further into the subject, and so that the tip is prevented from entering the dermis. This helps avoid contact with nerves, which can lead to pain.

In this configuration, the body 412.1 of the microstructure can be coated with a layer of insulating material (not shown), with only the tip exposed. As a result a current signal applied between the microstructures, will generate an electric field E within the subject, and in particular within the viable epidermis VE, so that measurements reflect fluid levels in the viable epidermis VE.

However, it will be appreciated that other configurations can be used. For example, in the arrangement of FIG. 4M, the shaft 412.14 is lengthened so the tip 412.2 enters the dermis, allowing dermal (and optional epidermal) measurements to be performed.

In this example, typical dimensions are shown in Table 2 below.

TABLE 2

| Parameter | Min. | Typical | Max. | Units |
|---|---|---|---|---|
| Length | 50 | 250 | 450 | microns |
| Width | 50 | 250 | 450 | microns |
| Thickness | 10 | 40 | 80 | microns |
| Density | 10 | 50 | 200 | cm⁻² |
| Tip radius | 0.1 | 2 | 10 | microns |
| Surface area | 10,000 | 62,500 | 427,000 | micron² |
| per electrode | | | | |
| Buttress width | 30 | 75 | 150 | microns |
| at base | | | | |

An example of the inter and intra pair spacing for these configurations are shown in Table 3 below.

TABLE 3

| Parameter | Min. | Typical | Max. | Units |
|---|---|---|---|---|
| Separation | 10 | 500 | 2000 | microns |
| between | | | | |
| microstructures | | | | |
| in a group or pair | | | | |
| Separation | 200 | 500 | 2000 | microns |
| between groups of | | | | |
| microstructures | | | | |

Specific example microprojection arrangements are shown in FIGS. 4N to 4Q. In this example, pairs of microstructures are mounted on mesas to facilitate controlling penetration of the microstructures into the epidermis. Dimensions in mm are shown in FIGS. 4P and 4Q.

Results of a penetration experiment using the above microstructures are shown in FIG. 4R. Specifically, in this example, a handheld force gauge was used to measure a constant force of ION, which was applied to the back of the patch and held for 10 seconds. 0.1 mL of Crystal Violet solution was administered to the application site, with excess solution being removed after 10 minutes and the application site was imaged using a bench top microscope. This highlights successful penetration of the stratum corneum.

An example of the process for monitoring hydration will now be described in more detail with reference to FIG. 5.

In this example, a patch including microstructures similar to those outlined above is applied to a subject at step 500, with bioimpedance measurements being performed at step 510, by applying an electrical signal between rows of microstructures and measuring the resulting response via the same microstructures. This is typically performed initially in order to establish a baseline, and hence is performed prior to any perturbation of fluid levels within the subject, for example performing this pre-physical exertion, although this is not essential. The bioimpedance measurements are typically performed at multiple frequencies, including at least one “low frequency” measurement, typically performed at 50 Hz or less, at least one “high frequency” measurement, typically performed at 10 kHz or more, and one or more “intermediate frequency” measurements, typically performed at about 100 Hz. Additionally, measurements may be performed using rows of microstructures with different spacings, to ensure bioimpedance measurements reflect the impedance of fluid levels at different depths within the viable epidermis and/or dermis.

At step 520, fluid levels and their relative compartmental distribution within the subject are perturbed, with this being performed in any appropriate manner. For example, this can include having the subject ingest or withhold fluids, physically exert themselves, undergo postural changes (which can induce shifts in fluid between different compartments), take medication, or the like.

Following this, further bioimpedance measurements are recorded at step 530. Whilst this is shown as a separate discrete step compared to step 510, as the patch is wearable, this is not necessarily the case, and in practice bioimpedance can be monitored continuously or substantially continuously (for example every few seconds). At step 540, details of the perturbation event are recorded, allowing this to be taken into account when analysing the bioimpedance measurements. This could be performed in any appropriate manner, for example by having a user (either the subject or an overseeing individual) enter details of the perturbation event, or by monitoring signals from one or more sensors. For example, changes in respiration, heart rate and/or temperature, could be used to determine if the user has commenced or ceased physical exertion, whilst orientation/movement sensors could be used to determine if the subject has undergone a postural shift, such as sitting, standing, or the like.

These processes could then be repeated as needed, for example monitoring over a series of perturbations, so that the system continuously captures bioimpedance changes as fluid levels within the subject are perturbed.

At step 550, the measured bioimpedances are analysed to monitor changes in bioimpedance, with these changes being used to generate and display an indicator at step 560, for example to indicate if the subject is over or under hydrated, their fluid levels are restoring, they are hydrated but trending towards dehydration, they have a maldistribution of fluid between compartments, or the like.

Examples of measurements performed on subjects will now be described. In this regard, preliminary evaluation of prototype wearable hydration sensors was performed in-house using healthy volunteers, with a mild exercise-dehydration protocol based on static cross trainer equipment, with responses being observed across the interrogated frequencies (10 Hz-200 kHz). Assessment of body water loss was through precise body weighs and urine specific gravity measures using a refractometer. All measures confirmed body water loss to a mean of 1.5% body mass and the physiological anti-diuretic response was confirmed by urine specific gravity reduction and subsequent restoration after oral rehydration. Sensor patches similar to those described above, including rows of microstructures, were applied to the non-dominant shoulder and the exertional activity was treadmill-like and involved only major trunk and leg muscles.

A protocol consisting of application of the sensor, settling time, sub-maximal exercise, rest and then rehydration was performed by 16 healthy subjects. Three subjects performed multiple exercise-recovery cycles without oral rehydration. A typical dataset from this protocol is presented in FIG. 6A, with equivalent surface based impedance measurements being shown in FIG. 6B. FIGS. 7A to 7S show an example of exercise-recovery-rehydration impedance profiles across a spectrum from 1 Hz to 1 MHz. Throughout these figures, the periods of exercise, rest and rehydration are as labelled in FIGS. 6A and 6B.

The results highlight there is a clear dynamic response to fluid shifts in exercise and rest phases. In the case of measurements made using a microstructure patch, similar patterns are observed across all trials, meaning the results are consistent. Additionally, this demonstrates there is no one stable measure of hydration, but rather that fluid (including blood) dynamically shifts with exertion and that signal changes can be large (>50%).

In contrast, for the surface based measurements shown in FIG. 6B, the data shows a rapid initial drop in impedance resulting from sweat build-up on the skin surface, with a high uniform conductivity remaining across recovery and rehydration phases until the electrodes begin drying more than 3 hours after exercise ceased.

This highlights that extracting a single estimate of body water is fraught with complexity in a dynamic system such as the human body's water response to and during physical exertion, particularly in water restricted environments. However, with the unique benefit high temporal resolution of the impedance measurements collected using the wearable microstructure patch, the rates of water transport between key compartments can be characterised. In this regard, there is a clear differentiation of fluid shift in the rest period post physical exertion between ECF (10 Hz—FIG. 7P) and ICF dominated response (100 kHz—FIG. 7D). Effects are clearly seen in all subjects when the sensor measures remotely to the region performing the physical exertion. Signal changes are observed in the shoulder when the trunk and lower body are physical exerted. Thus, monitoring differences in how ECF and ICF change post a perturbation can be used to characterise fluid movements between compartments, and assess whether fluid is being extracted from ICF, for example in the event of water being used during physical exertion, or restored to ICF post physical exertion.

An initial characterisation has been performed by fitting a linear approximation to the water depletion (physical exertion) periods D1, D2, D3 and water restoration (rest) periods for the pooled data. In this manner, biases in measuring actual impedances due to patch application variability and inter-subject conductivity variability can be avoided. The gradients of these responses are plotted in FIG. 8A, which shows a significant difference in fluid depletion characteristics as the subjects dehydrate. Furthermore, the restoration of ICF appears to occur in preference to ECF after physical exertion. On settling of the ICF curve, we then see restoration of the ECF which may be considered as a ‘conduit’ from plasma to intracellular compartments. These results are further supported by FIG. 8B, which show how gradients reduce after successive depletion events, corresponding to the subject becoming more dehydrated.

Thus, these results highlight that water is shifted from intracellular compartments in response to exertion, and that the response is observed remote from the region performing the work, meaning a global response to physical exertion is seen. This fluid shift response may include fluid moving to and from ICF via ECF to blood and vice versa. Additionally, a component of this response will be that regional blood flow changes for both nutrient and thermal management purposes. On recovery, fluid is restored to intracellular compartments from plasma, via extracellular environment, so that ICF will restore first, and then when the osmotic drag which drives this fluid diffusion is reduced, ECF will replenish, with the rates being dependent in part, on available body water.

Consequently, observing the relative changes in ICF and ECF can be used to understand whether the body is hydrated, dehydrated, in the process of dehydrating or undergoing restoration. For example, an increasing ICF/ECF ratio suggests water is moving into ICF, and hence that a subject is undergoing restoration, whereas decreasing ICF suggests water is being used by the body faster than it is being replenished, so the subject is using fluid. Furthermore, the reducing gradients demonstrate that the rate of fluid flow is decreasing, which can in turn be indicative of a subject becoming dehydrated.

Accordingly, the above described arrangement can penetrate into the skin and interrogate the live tissue of the epidermis and the dermis, specifically interrogating extracellular and intracellular fluid compartments, allowing fluid shifts between compartments to be monitored.

Modelling the bio-physical behaviour using equivalent electrical component can help deconvolve the measured impedance data for hydration related signals from confounders such as blood-pressure fluctuations, physiological changes, temperature changes, sweat, or combinations thereof.

In one example, this is achieved taking into account equivalent circuit models used to represent human bio-physical processes, with output from the models being used to derive hydration indicators as inputs to machine learning and inferential data science models.

The signal transduction used for dehydration monitoring relies on the hypothesis that fluid shifts between the extracellular (Interstitial fluid—ISF and Vascular fluid) and intracellular compartments can shift the tonicity of the ionic environment resulting to a measurable impedance change. Devices that are on-skin have the additional complexity of mitigating the large impedance offered by the stratum corneum (the outer more layer of the skin). By virtue of being in-skin the current arrangements can readily and continuously access these dynamic signals.

FIGS. 9A and 9B show multifrequency bio-impedance response curves for 10 Hz and 100 kHz capture during repeated periods of activity and rest. While the multifrequency bio-impedance technique records an entire spectrum of frequencies between 10 Hz and 100 kHz only two are presented here for explanation purposes. It is evident that the magnitude of the change for rest and workout is different at different frequencies. Similarly, gradients or rate of change of the magnitude in response to exertion or rest, is different at different frequencies. Consequently a hydration index may be derived from signal analysis, first or second differentials of signal changes, or the like. Thus, it will be apparent that at least in some example, the hydration indicator can be based on signals over time, for example using rate of change in impedance at different frequencies to indicate hydration.

In this regard, it is generally understood that measurements at different frequencies can differentially detect characteristics of extra-cellular fluid (ECF) and intra-cellular fluid (ICF). In this regard, impedance measurement at lower frequencies are largely measurements of ECF, and in particular, interstitial fluid, whilst measurements at higher frequencies are indicative of both the ECF and the ICF compartments. Accordingly, differences in, or changes over time in, impedance measurements at different frequencies can inform the hydration status of the human body.

In one example, a bioimpedance model is used that differentiates between the high and low frequency impedance response of tissue by considering two parallel arms, one representing the low frequency Extracellular fluid response and the other representing the higher frequency intracellular fluid response, as shown in FIG. 10. This model can also include further components to isolate confounding parameters, such as an interfacial capacitance, shown in FIG. 10, which represents the often large impedance dominating the lower frequencies of bio-impedance spectrums.

In this regard, the interfacial impedance or Electrode Polarization (EP) as it is better known, is a physical phenomenon that is fate accompli to metal-electrolyte interactions, has been studied well for over a century and is present in two-electrode systems employed to measure the impedance response of an ionic environment. EP manifest as a double layer capacitor comprising of counter-ions adsorbed onto the surface of the electrode with a diffuse layer of ions surrounding it, driven by the source AC signal. It shields the larger response of the ionic environment from being measured at low frequencies. Above a certain frequency the effect of this capacitance is lowered. However with limited bandwidth of frequencies available in compact electronic packaging it can be noticeable. Since EP is a capacitance on a Bode plot of Phase vs. Frequency this effect shows up at frequencies where the phase is between −90 and −45 degrees.

Preferably the EP effect is confined to as low a frequency regime as possible to ensure that a maximum of the frequency spectrum generated by the arrangements described herein is available for sensing changes in the ionic environment. For example, by employing a better signal generation and measurement system, the large EP effect shown at 1101 in FIG. 11A can be reduced significantly as shown in FIG. 11B, resulting in an improved effective measurement region 1102.

The in-vitro characterization of the measuring devices is important as it provides a baseline response of the sensor. This baseline response describes how the device would respond in the presence of a purely ionic environment and in the absence of any biological media. In-vitro characterization is carried out in solutions ranging from tap water up to 0.9% saline solutions. Physiologically relevant saline concentration stands at 0.9% corresponding to the ionic equivalent of blood plasma however tissue not readily serviced by blood vessels may encounter more dilute ionic conditions down to as low as 0.09%. Tap water is employed at the lowest end of the tonicity investigation instead of De-ionized (DI) water since the integrity of the later is hard to maintain without specialized equipment.

FIG. 12A shows the in-vitro response of an uncoated measuring device in solutions of differing salinity. A simple model comprising of a series Resistor-Capacitor, is use to fit this data, as shown in FIG. 12B, which are interpreted as solution resistance at the higher frequencies and EP (double layer capacitance) contribution at lower frequencies. However there exists at the higher frequencies a knee feature which remains unaccounted for by the chosen model. This knee is entirely an ionic capacitance and is related to high frequency charging and discharging of the electrolyte itself hence the addition of a small parallel capacitance helps in modelling this feature as shown in FIG. 12C. Every component added to a model needs to have a specific physical meaning and has to be verified by executing a change in the same. The small parallel capacitance is seen to decrease at higher concentrations thus ensuring that the effect is there. This fact is also verified from literature.

Any iteration of a sensor device will need to be analysed and verified in saline solution and its model ascertained. FIGS. 13A, 13D; 13B, 13E and 13C, 13F show three different models for the uncoated (13A, 13D), full Parylene coated (13B, 13E) and Parylene etched (13C, 13F) microstructures, in which the microstructures include Parylene coating on their base, while the tips are free of the coating and have the bare metal exposed. Each of these models represent physical properties of the device and attempts to interpret the resulting plots. The response for the full Parylene coated device is of interest in this regard. While it is expected to be a mostly capacitive response, given the low dielectric constant of the Parylene coating, there still exists a need for a parallel charge transfer resistance to describe the data adequately. Albeit this charge transfer resistance is quiet high (in Mega-Ohms) as expected. The series resistance in the model represents the solution resistance at high frequencies as before. Indeed in literature such models have been employed to describe fully coated electrodes in ionic solutions.

FIG. 13F depicts the response of a device that has a Parylene coating on its base while the tips are free of the coating and have the bare metal exposed. The response in the low frequencies is mostly capacitive and is represented by the series R-C unit of the model while the high frequencies where the situation represents that of the uncoated device a similar arm as that of FIG. 13A is used. Together this model serves as a good representation of the device data.

Temperature is a known confounder for impedance measurements and bioimpedance measurements are not immune to the same. An increase in temperature in ionic solutions tends to reduce internal resistance due to a thermal agitation effect coupled with a decrease in solution density. Similarly an increase in temperature also increases Capacitance by effecting the permittivity (dielectric constant) of the solution.

Anecdotally large swings in response have been observed as a result of this factor. Hence it is useful to assess in-vitro settings for the present architecture to identify what percentage of the response varied directly with temperature. An uncoated device was chosen for this investigation and introduced to tap water and solution of 0.09% saline separately. Both solutions were heated from a room temperature of about 24 C to 40 C and then cooled down to room temperature. It can be seen from the results of tap water shown in FIG. 14A that there is a change in impedance of the system as temperature changes. Using the model for the uncoated devices shown in FIG. 14B and introduced afore the resistive and capacitive components were extracted, allowing a temperature coefficient of resistance (denoted by a) to be calculated, as shown in FIG. 14C. It can be seen that for the given device type per degree change in resistance is less than 2%.

This allows regimes of passive and active dehydration to be used to build models above the baseline ones presented above for the various device architectures.

From in-vivo experiments similar to those described above with respect to FIGS. 9A and 9B, it is evident that changes in the ionic environment are being picked up by the present devices at different frequencies. By employing the biophysical models, this can be used to extract data that most correlates to a hydration measure.

In this regard, the metal electrode-biological tissue system has two impedance dispersion regions in its response. The first is depicted as the alpha-dispersion and is related to the capacitance of the double layer in the lower frequencies. As the drive frequency is increased a second beta-dispersion is observed which is characteristic of the capacitance of cellular membranes. Above this frequency threshold the impedance response contains the sum of both intra and extra cellular components. In literature for different measurement architectures different frequencies are identified for the beta-dispersion but most are above 50 KHz. On the contrary a metal electrode-electrolyte system like that encountered in-vitro experiments where a device is introduced to a saline solution, will not have a beta-dispersion owing to there being no cellular media present. This can be seen in FIGS. 15A and 15C where after the initial drop in impedance (low frequencies) the response becomes flat in the higher frequencies. On the other hand in FIGS. 15B and 15D the impedance can be seen to drop with slight variation in the lower frequencies and then again in the higher frequencies indicating that with further assessment it may be possible to realize the model depicted in FIGS. 15E and 15F allowing the intra and extra cellular components of the fluid compartments to be analyzed in-skin.

Further investigations also include the assessment of different layers of the skin that have either been penetrated by the microstructures, or form part of the data received due to the fringing field effect present in such devices, allowing a more complex model such layer by layer investigation could be carried out given known conductivities of the different layers which convert through trivial relations to Resistance values specific to device dimensions.

### Human Trials

Wearable sensors, with micro-projections that penetrate the skin to access physiological data, have been tested extensively in vitro, in ex vivo animal models and human interfacing experiments. Furthermore, preclinical work and finite element simulations demonstrate that blade micro-electrodes of appropriate size and structure can help to concentrate the working electric field into a target skin layer, improving the accuracy and reproducibility of measurements.

Surface electrodes, which is the basis of most of present commercial and academic embodiments for hydration related characterization, are significantly influenced by the hydration of the stratum corneum, which changes significantly with the environmental conditions, and influenced further with body sweat.

A comparison of the measured impedance for surface-based electrodes (on-skin measurements) and blade microstructure electrodes in the epidermis (in-skin) is shown in FIGS. 16A and 16B. Here electrodes spaced 1 mm apart are placed onto dry skin, skin with mild to moderate perspiration and skin with heavy perspiration, where the perspiration is simulated with a saline aerosol. Impedance frequency sweeps of surface electrodes on the skin demonstrate a 500× difference in impedance as a function of sweat on the stratum corneum. Impedance frequency sweeps of the microstructure electrode device applied to the arm, which interrogates within the epidermis only and bypasses the stratum corneum, demonstrates a much smaller impedance change.

The functional location of water within the body is conceptually explained in terms of compartments. Water can be categorised as intracellular or extracellular (inside or outside of cells respectively), with the extracellular fluid compartment further broken up into the interstitial fluid (ISF) and plasma fluid compartments. Water in this ISF compartment is a fluid reservoir which dynamically increases and decreases in volume as the body maintains water homeostasis. The goal is to maintain plasma osmolarity and volume to ensure perfusion of essential organs such as heart, brain, lungs and kidney and is achieved in the first instance by water shifting out of the ISF. This phenomenon is exploited by the sensing approach to allow early and sensitive indications of body water status.

Since ISF plays a key role in compensating for fluid loss under heat-induced dehydration conditions, this physiological response of skin water content is exploited as an effective way to measure overall hydration.

The present arrangement employs multi-frequency bio-impedance technique to measure minute changes in the skin's electrical properties, which may be related to fluid shifts in internal compartments and eventually may inform the body hydration.

Trials have been employed to identify a hydration signal using a microstructure sensing patch applied to human subjects undergoing short-intervals of high-intensity workout sessions (5 mins each) carried out between parallel lines in the plots interspaced by a longer rest interval. Over many experiments it has been shown that a repeatable and reliable response can be achieved from the hydration prototype which seems to reflect exercise and rest intervals.

Extensive preclinical studies of exemplary devices have been undertaken, including a pre-pilot human experiment in a controlled environment, allowing small changes from within the skin to be measured. To initiate detectable signal changes the subject exercised in an environmental chamber to induced dehydration, with detectable fluid shifts in the skin which demonstrated the utility of the sensor platform. This pre-pilot study allowed for functional testing of prototype hydration sensors.

The trial work involved the subject being actively dehydrated through exercise in an environmental chamber over the course of several hours (weight loss of 3.3%). Six devices were used to measure skin impedance. The sensors used in this study include 30 stainless steel microneedles of 300 μm length, affixed to the body with tape. The device needles painlessly penetrate the skin to a depth of approximately 150 μm. Surface sensors having 30 blunt stainless-steel microneedles (acupuncture needles) were designed not to penetrate the skin were also affixed to the body with tape. All sensors were powered by 3.7V 400 mAh LiPo batteries. Impedance spectrums were recorded between 10 Hz-100 kHz across the two separations of the sensors (1.0 mm and 2.0 mm) at 24 discreet frequencies, every 45 seconds, yielding 384 impedance measures every 45 seconds. Additional parameters recorded during the experiment were environmental humidity and temperature, core body and skin temperature, heart rate and weight. The devices and the corresponding recording hardware were applied on both arms and shoulders of the subject.

The experimental protocol in short was as follows: Device application [symbol] Subject enter the environmental chamber [symbol] 5 min baseline measurement [symbol] 45 min exercise [symbol] exit chamber, blood draw, naked weigh-in after towel drying [symbol] re-enter chamber [symbol] 45 min of exercise [symbol] exit chamber, blood draw, naked weigh-in after towel drying [symbol] re-enter chamber [symbol] 45 min of exercise [symbol] exit chamber, blood draw, naked weigh-in after towel drying [symbol] rehydrate for 75 min [symbol] device removal.

Examples of raw impedance measurements are shown in FIG. 17, highlighting changes in impedance during and post exercise.

Preliminary visual analysis of the results reveals the following:
Impedance significantly drops 10 min into exercise
Impedance increases ˜6 min into both recovery periods
Impedance decreases minutes after entering environmental chamber following rest periods
At low frequencies, normalized magnitude changes are consistent between devices. At higher frequencies, there is greater variation of magnitude changes between devices.

Following this, further investigations were performed to separate hydration related signals from confounding and physiological effects, including the presence of varying temperature and varying blood pressure. In this regard, it is important to individually assess confounding factors that may occur during a hydration event as closely as possible to gain a better understanding of our overall signal.

To achieve this, hot and cold packs were used to heat and cool the skin surrounding the sensing devices, without changing the temperature of the devices themselves, in order to simulate natural body temperature changes an individual would expect to be subject to during physical activity, without having to introduce possible motion artefacts and changes caused by physical activity. Impedance measured at various frequencies on the dorsal hand with hot and cold packs applied for 10 minutes each, and each followed by a 10 minute recovery period with no applied heating or cooling are shown in FIGS. 18A and 18B. Initial data suggests temperature may not impact impedance measurements.

Further trials were used to assess uncoated and etched microstructures and surface electrodes under a variety of conditions, including during inactive/no sweat (seated in air con), inactive/sweat (seated in a heated greenhouse), and active/sweat (elliptical activity in heated greenhouse) conditions, including temperature and blood pressure confounding factor intervention. One of each sensor type was applied to four body sites for comparison (proximal upper arm, shin, hand, and sternum). Temperature was manipulated during the inactive/no sweat phase by applying hot and cold packs to application sites as per confounding factor studies. Blood pressure was changed by changing the participant's position from seated, to lying down, to standing up, and was measured with a blood pressure arm cuff at 2 minute intervals.

Results in FIGS. 19A to 19F show a more reliable signal from microstructure based sensors compared to on-skin impedance measurements performed with surface electrodes (FIGS. 19E and 19F), and some sweat insulation due to parylene coating (FIGS. 19C and 19D) after onset of exposure to a hot greenroom and activity. Etched devices had majority deformities due to poor gold adhesion in this particular batch.

Accordingly, the above described arrangements describe the use of microwearable patches that can be used to monitor hydration, with changes in bioimpedance following a perturbation event being used to analyse fluid shifts within a subject, and thereby provide feedback regarding a hydration state.

However, it will be appreciated that monitoring changes in impedance is not essential and alternatively static values of impedance from a single time point could be used.

Accordingly, in another example, a system for monitoring a fluid status of a biological subject, the system includes at least one substrate including a plurality of microstructures including electrodes configured to breach a stratum corneum of the subject, a signal generator configured to apply an electrical stimulatory signal between electrodes on different microstructures, at least one signal sensor configured to measure electrical response signals between electrodes on different microstructures and one or more electronic processing devices that are configured to determine one or more bioimpedance values using the measured electrical response signals and analyse the one or more bioimpedance values to determine at least one indicator at least partially indicative of the fluid status of the subject.

In such an arrangement, the one or more bioimpedance values could be measured at a single frequency, but more typically would use measurements at different frequencies in order to ascertain a fluid status. Thus, for example, measurements at low and high frequencies could be used to determine relative amounts of intra-cellular and extra-cellular fluid levels, which could in turn be used to derive a fluid status indicator.

In one example, such patches are 1 cm² devices, applied to the torso as an adhesive patch. Electrical addressing of penetrating electrodes is achieved with on-board electronics and wireless transmission to a display and archive tool such as a tablet or personal computer.

Trials of devices show responses which characterise physical exertion, recovery and re-hydration periods. As the patches are wearable, a high temporal resolution is possible, which in turn allows for monitoring of the dynamics of shifts in body water from and to plasma, ECF and ICF compartments (at least).

This platform can be the basis for a wearable hydration assessment tool and can also allow real-time analysis of body water dynamics to a) better understand the physiology of exertion in water-stressed environments, and b) provide personalised performance management of individuals undergoing activities, such as warfighters in preparatory activities, performance of tasks and in the recovery phases of missions. In one example, the patch and associated reader are enabled as an IoT (Internet of Things) connected device, and sharing of data can be at the discretion of the owner and users. The value of this data is realised in the personal hydration management of the individual and the benefits of pooled, anonymised data from large cohorts.

In this regard, due to the immense physical demand of their work, military personnel are more at risk of dehydration, and relatedly, heat illnesses. These avoidable conditions impact severely upon the ability to complete missions safely and effectively. Dehydration mediates its detrimental effects physically, cognitively and psychologically.

Dehydration severely impacts physical performance. For example, heat and water loss are intrinsically linked, so a warfighter working in a hot climate is more likely to become rapidly dehydrated, which, in turn, increases their risk of succumbing to heat stroke. The body's core temperature increases by 0.1-0.2° C. with every 1% body mass loss through dehydration. This is because water plays an important role in temperature regulation through the cooling effect of sweating. However, as sweating is water loss, the deficit must be addressed adequately and quickly through water intake to prevent dehydration. Physical symptoms present on a sliding scale of severity from headache, lethargy, dry mucosae and eyes and breathlessness in early stages to muscle spasms and hypovolemic syncope. The effects can be rapid, and 1-2% total body water loss can affect cardiovascular and thermoregulatory mechanisms sufficiently to perceive the requirement of extra effort, diminishing physical performance. If unaddressed, dehydration leads to death directly, or indirectly through reduced physical or mental capability. Impacts of physical incapacitation through dehydration (with and without heat and exertion) have been well characterised, primarily in healthy athletes and the military. A meta-analysis on its impact on physical ability demonstrated a marked impact of dehydration upon muscle strength (−5.5% vs hydrated), endurance (−8.3%), anaerobic power (−5.8%) and capacity (−3.5%). An active hydration procedure, i.e. featuring exertion, was associated with a 2.8-fold higher impact on performance than a passive one employing heat stress/fluid restriction only. One study proposed a threshold of 2% body weight loss through dehydration below which endurance and strength was significantly impaired. This level of dehydration may occur in as little as a few minutes with physical demand in a hot climate and so may compromise the mission almost from its outset.

Furthermore, even mild dehydration significantly reduces cognitive capabilities. For example, one of the first consequences of dehydration is a limitation in the availability of the tissue fluid perfusing the brain, resulting in changes to its structure and function. With a reduced fluid perfusion, the brain's volume shrinks significantly, as does that of the key cortical structures responsible for cognitive processes. Headache is a common neurological complaint of dehydration, but potentially more serious impacts in the form of behavioural and cognitive impacts which coincide with as little as a 1-2% total body water loss, may be less obvious to detect. In less severe cases, cognitive effects present as immediate memory loss, attention deficit, perceived task difficulty and reduction in visuospatial awareness but may proceed rapidly to severe confusion and disorientation if dehydration is not corrected. Cognitive impacts are significantly pronounced in the heat. In the field, any such reduction in alertness can cause critical delays in reaction time and inadvertent risk-taking endangering both individual and team. Many trials have formally linked dehydration with negative impacts on cognition. In one simulated task experiment, mildly dehydrated drivers were found to make as many errors as drivers who were sleep deprived, or drivers who had ingested alcohol equivalent to the legal limit for driving. In military personnel, 11% of aviators completed their scheduled flight with a fluid deficit greater than 1% despite a regular intake, demonstrating this level of dehydration may be common during military activities requiring an exceptionally high level of focus.

Dehydration also contributes to psychological stress. In this regard, dehydration is perhaps the most fundamental cause of stress in the body. When a water deficit is detected, potent neural-hormonal mechanisms are initiated to prompt fluid intake to prevent further damage to the body. Studies seeking to identify biological indicators of fluid status have shown an increase in serum cortisol levels with dehydration that returned to normal with rehydration. Cortisol is a neurotransmitter involved in the acute response to stress and is commonly increased in states of anxiety and panic. Dehydration-induced hypercortisolaemia has been proposed by some to be one cause of the impairment of active learning, short term memory and other cognitive impacts described above. Trials have commonly linked dehydration with reported psychological effects of anxiety and low mood. Even after a fluid restriction protocol of only 90 minutes, volunteers in one dehydration study reported low mood and anxiety alongside thirst sensation and decline in energy, that were subsequently reversible on rehydration. Neurotransmitters such as those required for maintenance of brain health require adequate water for synthesis and transport from their site of production to the site of action. In animal studies, a link has been made between dehydration and low levels of serotonin (a known cause of depression), via an inability to transport its precursor tryptophan across the brain to where it is required. A strong link between dehydration and long-term mental illness is yet to be formally accepted, though one large-scale study has found association between fluid status and depression score. In summary, adequate hydration is a vital contributor to optimal psychological resilience in the face of high levels of acute stress as experienced in combat environments.

In the case of military personnel, severe dehydration may result in unscheduled on-mission IV rehydration stops, slowing soldiers and delaying the mission, or in extreme cases, requiring relocation to a field medical centre. The list of resources expended on these activities includes not just time and effort diverted away from mission objectives, but a requirement for logistics support to transport IV rehydration equipment or in the latter case, hospital repatriation costs.

A ‘Personalised Hydration Plan’ tailored to the individual's physiology could prevent either of these situations from occurring by allowing for earlier, more improved control over the unknowns of hydration during a mission. As one US Army medic wrote: “Arguably the most important part [of staying hydrated] starts before they ever set foot on mission: pre-hydration or drinking plenty of fluid and eating well on the day(s) prior. You can't be dehydrated and play catch-up during a physical event. Unfortunately for last minute calls and responses, this isn't always easy to prepare for”. Such forward planning could ensure the correct action for full recovery, as drinking ad libitum in response to thirst often falls short of the amount required to fully rehydrate, resulting in a deficit carry-over impacting performance for days afterward. In addition to planning and recovery, real-time monitoring is vital to success, to allow deviations with changing environments. Despite the urgent need for better ways to monitor fluid status accurately and in real time, no such solution exists.

The above described arrangements provide a wearable hydration monitor for real-time on-person hydration monitoring in the field. Recent reviews have illustrated the need for hydration monitoring technology suitable for field use. Many heat-stressed, exertional occupations such as military operations can benefit as discussed above. Among the candidate measures, multifrequency bioimpedance shows promise, but lacks the desired sensitivity and specificity primarily due to interrogation of bulk body tissue using surface electrodes across the surface of the skin. To overcome the insulating properties of the outer stratum corneum and target cellular components and ECF in a minimally invasive fashion, arrays of microstructures are fabricated which are electrically addressable and able to be applied by hand. Due to the shallow penetration the devices are pain free and do not cause bleeding nor induce local erythema.

Practical implementations of the sensor patch and electronics have been developed and can be worn for prolonged periods (˜24 hours). Interrogation can be via Near Field Communications (NFC) protocols which are able to be used in smartphones and for which numerous existing reader solutions are available. The NFC system can activate the custom programmed integrated circuit via a radio frequency induction coil. A reader can then be used to provide instantaneous measures of impedance and allows basic signal processing and storage with the option of cloud telemetry. In this way, the system provides an Internet of Things (IoT) solution with data access being subject to the usual permissions and security implementations.

Body water is well recognised as being present in conceptual compartments, principally extracellular, which includes blood and plasma and intracellular. The electrical properties of these tissue types are measurable and can be modelled with a lumped-constant model—typically the Cole-Cole Model. Essentially, capacitive components in the complex impedance are due to intracellular water and the ionic ECF is principally the parallel resistive component. Discrimination of water content in different tissue types (compartments) can then be performed using multifrequency approaches—in the first instance a simple low frequency—high frequency discrimination demonstrates the proof-of-concept.

In one example, the above described system allows fluid measurements, such as ion concentration and/or hydration measurements to be performed. The length of the structures can be controlled during manufacture to enable targeting of specific layers in the target tissue. In one example, this is performed to target fluid levels in the epidermal and/or dermal ISF.

The patches can therefore provide a measurement device which avoids the need to perform surface based measurements, allowing measurements to be performed that are more accurate and/or sensitive.

The system can provide simple, semi-continuous or continuous monitoring: a low cost-device micro wearable would be applied to the skin and potentially be worn for days (or longer), and then simply replaced by another micro wearable component. Thus, micro wearables provide a route for monitoring over time—which can be particularly important in circumstances where fluid levels are changing rapidly.

In one example, the above described approach can allow wearables to provide widespread, low-cost healthcare monitoring for a multitude of health conditions that cannot be assayed by current devices, which are placed on the skin.

Whilst the above examples illustrate the importance of monitoring fluid levels in military applications, it will be appreciated that monitoring fluid levels is equally applicable in a range of different scenarios, for example in monitoring elderly people, athletes, workers in extreme and particular heat stressful environments, patients in a medical context, or the like. Similarly, whilst the above has focused on use of the device in assessing hydration, it will be appreciated that the device and associated analysis can be used for monitoring fluid status more broadly for a wide range of different purposes, including, for example, monitoring fluid levels for controlling dialysis, monitoring fluid levels in a post-operative procedure, monitoring fluid levels when a subject is undergoing vomiting/diarrhoea, when administering IV fluid or diuretics, and for monitoring patients undergoing, or at risk of renal failure, heart failure, or the like.

Accordingly, it will be appreciated that the term subject can include living subjects, such as humans, animals, or plants, as well as non-living materials, such as foodstuffs, packaging, or the like.

Accordingly, the above described arrangement provides a wearable monitoring device that uses microstructures that breach a barrier, such as penetrating into the stratum corneum in order to perform measurements on a subject. The measurements can be of any appropriate form, and can include measuring the fluid levels within the subject, measuring electrical signals within the subject, or the like. Measurements can then be analysed and used to generate an indicator indicative of a health status of the subject.

Persons skilled in the art will appreciate that numerous variations and modifications will become apparent. All such variations and modifications which become apparent to persons skilled in the art, should be considered to fall within the spirit and scope that the invention broadly appearing before described.

1) A system for monitoring a fluid status of a biological subject, the system including:
a) at least one substrate including a plurality of microstructures including electrodes configured to breach a stratum corneum of the subject;
b) a signal generator configured to apply an electrical stimulatory signal between electrodes on different microstructures;
c) at least one signal sensor configured to measure electrical response signals between electrodes on different microstructures; and,
d) one or more electronic processing devices that are configured to:
i) determine changes in bioimpedance using the measured electrical response signals; and,
ii) analyse the changes in bioimpedance to determine at least one indicator at least partially indicative of the fluid status of the subject.

2) A system according to claim 1, wherein at least one of:
a) the bioimpedance is at least one of:
i) measured at a single frequency;
ii) measured at multiple different frequencies; and,
iii) derived from impedance measurements performed at multiple different frequencies; and,
b) the bioimpedance is indicative of at least one of:
i) intracellular fluid levels;
ii) extracellular fluid levels; and,
iii) blood fluid levels.

3) (canceled)

4) A system according to claim 1, wherein the change in bioimpedance includes at least one of:
a) a change in a bioimpedance magnitude;
b) a change in a bioimpedance phase angle;
c) a change in intracellular fluid levels;
d) a change in extracellular fluid levels; and,
e) a change in blood fluid levels.

5) A system according to claim 1, wherein the one or more electronic processing devices are configured to:
a) analyse changes in bioimpedance to determine fluid movement between fluid compartments; and,
b) generate the indicator based on the determined fluid movement.

6) A system according to claim 1, wherein the one or more electronic processing devices are configured to:
a) determine a baseline bioimpedance; and,
b) analyse changes in bioimpedance relative to the baseline bioimpedance.

7) A system according to claim 1, wherein the one or more electronic processing devices are configured to:
a) determine a perturbation event that will perturb fluid levels in the subject; and,
b) analyse the changes in bioimpedance at least in part in accordance with the perturbation event.

8) A system according to claim 7, wherein at least one of:
a) the perturbation event includes at least one of:
i) a change in physical activity state;
ii) a change in posture;
iii) heating;
iv) cooling;
v) ingestion of fluid;
vi) administration of medication;
vii) administration of a pharmacological agent;
viii) a medical procedure;
ix) dialysis;
x) administration of intravenous fluids;
x) administration of intravenous blood;
xii) onset of illness or disease; and,
xiii) a physiological perturbation; and,
b) the one or more electronic processing devices are configured to at least one of:
i) determine a change in bioimpedance measured before and after the perturbation event;
ii) determine a change in bioimpedance measured during the perturbation event;
iii) determine a change in bioimpedance during a time period after the perturbation event; and,
iv) determine a rate of change in bioimpedance during a time period after the perturbation event;
v) compare multiple changes in bioimpedance, each change in bioimpedance being associated with a respective perturbation event and determine the indicator based on the multiple changes in bioimpedance; and,
vi) determine a gradient of a rate of change in bioimpedance after each of multiple perturbation events and determine the indicator based on the changes in the gradients:
c) the one or more electronic processing devices are configured to determine the perturbation event based on at least one of:
i) user input commands;
ii) signals from at least one sensor:
iii) changes in a subject movement;
iv) changes in a subject posture;
v) changes in a subject temperature;
vi) changes in a subject heart rate;
vii) changes in a subject respiratory rate; and,
viii) changes in a subject blood oxygen levels.

9) (canceled)

10) (canceled)

11) (canceled)

12) (canceled)

13) A system according to claim 7, wherein the system includes a sensor at least one of:
a) mounted on the substrate; and,
b) provided within a housing attached to the substrate, and wherein the one or more processing devices are configured to:
i) monitor sensor signals from the at least one sensor; and,
ii) determine the perturbation event in accordance with the sensor signals.

14) A system according to claim 1, wherein the indicator is indicative of at least one of:
a) over hydration;
b) under hydration;
c) normal hydration;
d) restoration;
e) trending towards dehydration; and,
f) maldistribution of fluid between compartments.

15) A system according to claim 1, wherein at least one of:
a) the microstructures are arranged in pairs and wherein the bioimpedance is measured using at least one of:
i) multiple pairs of electrodes; and,
ii) pairs of electrodes with different spacings; and,
b) the microstructures are arranged in rows and wherein the bioimpedance is measured between at least one of:
i) electrodes on different rows of microstructures; and,
ii) electrodes on different rows of microstructures with different spacings,
c) at least some of the microstructures are blade microstructures.

16) (canceled)

17) A system according to claim 1, wherein at least one of:
a) a spacing between the microstructures is at least one of:
i) about 2 mm;
ii) about 1 mm;
iii) about 0.5 mm;
iv) about 0.2 mm; and,
v) about 0.1 mm;
b) at least some of the microstructures at least one of:
i) are at least partially tapered and have a substantially rounded rectangular cross sectional shape;
ii) have a length that is at least one of:
(1) less than 300 μm;
(2) about 150 μm;
(3) greater than 100 μm; and,
(4) greater than 50 μm;
iii) have a maximum width that is at least one of:
(1) of a similar order of magnitude to the length;
(2) greater than the length;
(3) about the same as the length;
(4) less than 300 μm;
(5) about 150 μm; and,
(6) greater than 50 μm; and,
iv) have a thickness that is at least one of:
(1) less than the width;
(2) significantly less than the width;
(3) of a smaller order of magnitude to the length;
(4) less than 100 μm;
(5) about 25 μm; and,
(6) greater than 10 μm;
v) have a tip that at least one of:
(1) has a length that is at least one of:
(a) less than 50% of a length of the microstructure;
(b) at least 10% of a length of the microstructure; and,
(c) about 30% of a length of the microstructure; and,
(2) has a sharpness of at least one of:
(a) at least 0.1 μm;
(b) less than 5 μm; and,
(c) about 1 μm; and,
vi) include at least one of:
(1) a shoulder that is configured to abut against the stratum corneum to control a depth of penetration;
(2) a shaft extending from a shoulder to the tip, the shaft being configured to control a position of the tip in the subject; and,
(3) anchor microstructures used to anchor the substrate to the subject; and,
c) the microstructures have a density that is at least one of:
i) less than 5000 per cm²;
ii) greater than 100 per cm²; and,
iii) about 600 per cm².

18) (canceled)

19) (canceled)

20) (canceled)

21) (canceled)

22) A system according to claim 1, wherein at least one of:
a) the substrate includes electrical connections to allow electrical signals to be applied to and/or received from respective microstructures;
b) the system includes one or more switches for selectively connecting at least one of the at least one sensor and at least one signal generator to one or more of the microstructures and wherein the one or more processing devices are configured to control the switches and the signal generator to allow at least one measurement to be performed;
c) the system includes:
i) a substrate coil positioned on the substrate and operatively coupled to one or more microstructure electrodes; and,
ii) an excitation and receiving coil positioned in proximity to the substrate coil such that alteration of a drive signal applied to the excitation and receiving coil acts as a response signal.

23) (canceled)

24) (canceled)

25) A system according to claim 1, wherein the microstructures include an insulating layer extending over at least one of:
a) part of a surface of the microstructure;
b) a proximal end of the microstructure;
c) at least half of a length of the microstructure;
d) about 90 μm of a proximal end of the microstructure; and,
e) at least part of a tip portion of the microstructure.

26) A system according to claim 1, wherein at least one electrode at least one of:
a) has a surface area of at least one of:
i) less than 200,000 μm²;
ii) about 22,500 μm²; and,
iii) at least 2,000 μm²;
b) extends over a length of a distal portion of the microstructure;
c) extends over a length of a portion of the microstructure spaced from the tip;
d) is positioned proximate a distal end of the microstructure;
e) is positioned proximate a tip of the microstructure;
f) extends over at least 25% of a length of the microstructure;
g) extends over less than 50% of a length of the microstructure;
h) extends over about 60 μm of the microstructure; and,
i) is configured to be positioned in a viable epidermis of the subject in use.

27) A system according to claim 1, wherein the microstructures at least one of:
a) include a material including at least one of:
i) a material to reduce biofouling;
ii) a material to attract at least one substance to the microstructures; and,
iii) a material to repel at least one substance from the microstructures; and,
b) at least some of the microstructures are coated with a coating and wherein the coating at least one of:
i) modifies surface properties to at least one of:
(1) increase hydrophilicity;
(2) increase hydrophobicity; and,
(3) minimize biofouling;
ii) attracts at least one substance to the microstructures;
iii) repels at least one substance from the microstructures;
iv) acts as a barrier to preclude at least one substance from the microstructures; and,
v) includes at least one of:
(1) a permeable membrane;
(2) polyethylene;
(3) polyethylene glycol;
(4) polyethylene oxide;
(5) zwitterions;
(6) peptides;
(7) hydrogels; and,
(8) self-assembled monolayer.

28) (canceled)

29) A system according to claim 1, wherein the system includes:
a) a patch including the substrate and microstructures; and,
b) a monitoring device that is configured to:
i) perform the measurements; and,
ii) at least one of:
(1) provide an output indicative of the indicator; and,
(2) provide a recommendation based on the indicator.

30) (canceled)

31) A system according to claim 1, wherein the system includes:
a) a transmitter that transmits at least one of:
i) subject data derived from the measured response signals; and,
ii) measured response signals; and,
b) a processing system that:
i) receives subject data derived from the measured response signals; and,
ii) analyses the subject data to generate at least one indicator, the at least one indicator being at least partially indicative of a health status associated with the subject.

32) A system according to claim 1, wherein the system is configured to perform impedance measurements in the viable epidermis to determine an indicator indicative of at least one of:
a) a hydration of the subject;
b) interstitial fluid levels;
c) a change in interstitial fluid levels;
d) an ion concentration in interstitial fluid;
e) a change in an ion concentration in interstitial fluid;
f) an ion concentration;
g) a change in an ion concentration;
h) a total body water;
i) intracellular fluid levels;
j) extracellular fluid levels;
k) plasma water levels;
l) fluid volumes; and,
m) hydration levels.

33) A method for monitoring a fluid status of a biological subject, the method including:
a) providing:
i) at least one substrate including a plurality of microstructures including electrodes configured to breach a stratum corneum of the subject;
ii) a signal generator configured to apply an electrical stimulatory signal between electrodes on different microstructures; and,
iii) at least one signal sensor configured to measure electrical response signals between electrodes on different microstructures; and,
b) using one or more electronic processing devices to:
i) determine changes in bioimpedance using the measured electrical response signals; and,
ii) analyse the changes in bioimpedance to determine at least one indicator at least partially indicative of the fluid status of the subject.

34) A system for monitoring a fluid status of a biological subject, the system including:
a) at least one substrate including a plurality of microstructures including electrodes configured to breach a stratum corneum of the subject;
b) a signal generator configured to apply an electrical stimulatory signal between electrodes on different microstructures;
c) at least one signal sensor configured to measure electrical response signals between electrodes on different microstructures; and,
d) one or more electronic processing devices that are configured to:
i) determine one or more bioimpedance values using the measured electrical response signals; and,
ii) analyse the one or more bioimpedance values to determine at least one indicator at least partially indicative of the fluid status of the subject.

35) A method for monitoring a fluid status of a biological subject, the method including:
a) providing:
i) at least one substrate including a plurality of microstructures including electrodes configured to breach a stratum corneum of the subject;
ii) a signal generator configured to apply an electrical stimulatory signal between electrodes on different microstructures; and,
iii) at least one signal sensor configured to measure electrical response signals between electrodes on different microstructures; and,
b) using one or more electronic processing devices to:
i) determine one or more bioimpedance values using the measured electrical response signals; and,
ii) analyse the one or more bioimpedance values to determine at least one indicator at least partially indicative of the fluid status of the subject.
